SEQUENCE LISTING (24 sequences)

SEQ ID NO: 1 - protein, 360 aa, Homo sapiens
MNPTDIADTTLDESIYSNYYLYESIPKPCTKEGIKAFGELFLPPLYSLVFVFGLLGNSVVVLVLFKYKRLRSMTDVYLLNLAISDLLFVFSLPFWGYYAADQWVFGLGLCKMISWMYLVGFYSGIFFVMLMSIDRYLAIVHAVFSLRARTLTYGVITSLATWSVAVFASLPGFLFSTCYTERNHTYCKTKYSLNSTTWKVLSSLEINILGLVIPLGIMLFCYSMIIRTLQHCKNEKKNKAVKMIFAVVVLFLGFWTPYNIVLFLETLVELEVLQDCTFERYLDYAIQATETLAFVHCCLNPIIYFFLGEKFRKYILQLFKTCRGLFVLCQYCGLLQIYSADTPSSSYTQSTMDHDLHDAL

SEQ ID NO: 2 - protein, 5 aa, Mus musculus
NYGMS

SEQ ID NO: 3 - protein, 17 aa, Mus musculus
TISSASTYSYYPDSVKG

SEQ ID NO: 4 - protein, 10 aa, Mus musculus
HSDGNFAFGY

SEQ ID NO: 5 - protein, 16 aa, Mus musculus
RSSRNIVHINGDTYLE

SEQ ID NO: 6 - protein, 7 aa, Mus musculus
KVSNRFS

SEQ ID NO: 7 - protein, 9 aa, Mus musculus
FQGSLLFWT

SEQ ID NO: 8 - protein, 119 aa, Mus musculus
EVQLVESGGDLMKPGGSLKISCAASGFIFSNYGMSWVRQTPDMRLEWVATISSASTYSYYPDSVKGRFTISRDNAENSLYLQMNSLRSEDTGIYYCGRHSDGNFAFGYWGRGTLVTVSA

SEQ ID NO: 9 - protein, 113 aa, Mus musculus
DVLMTQTPLSLPVSLGDQASISCRSSRNIVHINGDTYLEWYLQRPGQSPKLLIYKVSNRFSGVPDRFSGSGSGTDFTLKISRVEAEDLGVYYCFQGSLLPWTFGGGTRLEIRR

SEQ ID NO: 10 - protein, 119 aa, Artificial Sequence
  Description of Artificial Sequence Synthetic Peptide
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCARHSDGNFAFGYWGQGTLVTVSS

SEQ ID NO: 11 - protein, 119 aa, Artificial Sequence
  Description of Artificial Sequence Synthetic Peptide
EVQLVESGGGLVQPGGSLRLSCAASGFTFSNYGMSWVRQAPGKGLEWVSTISSASTYSYYPDSVKGRFTISRDNSKNTLYLQMNSLRAEDTAVYYCARHSDGNFAFGYWGQGTLVTVSS

SEQ ID NO: 12 - protein, 112 aa, Artificial Sequence
  Description of Artificial Sequence Synthetic Peptide
DIVMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIK

SEQ ID NO: 13 - protein, 119 aa, Artificial Sequence
  Description of Artificial Sequence Synthetic Peptide
EVQLVESGGDLVQPGRSLRLSCAASGFIFSNYGMSWVRQAPGKGLEWVATISSASTYSYYPDSVKGRFTISRDNAKNSLYLQMNSLRVEDTALYYCGRHSDGNFAFGYWGQGTLVTVSS

SEQ ID NO: 14 - protein, 119 aa, Artificial Sequence
  Description of Artificial Sequence Synthetic Peptide
EVQLVESGGDLVQPGRSLRLSCAASGFIFSNYGMSWVRQAPDKRLEWVATISSASTYSYYPDSVKGRFTISRDNAKNSLYLQMNSLRVEDTALYYCARHSDGNFAFGYWGQGTLVTVSS

SEQ ID NO: 15 - protein, 119 aa, Artificial Sequence
  Description of Artificial Sequence Synthetic Peptide
EVQLVESGGDLVQPGRSLRLSCAASGFIFSNYGMSWVRQAPDKRLEWVATISSASTYSYYPDSVKGRFTISRDNAKNSLYLQMNSLRVEDTALYYCGRHSDGNFAFGYWGQGTLVTVSS

SEQ ID NO: 16 - protein, 119 aa, Artificial Sequence
  Description of Artificial Sequence Synthetic Peptide
EVQLVESGGGLVQPGGSLRLSCAASGFIFSNYGMSWVRQAPGKGLEWVSTISSASTYSYYPDSVKGRFTISRDNSKNTLYLQMNSLRAEDTAVYYCARHSDGNFAFGYWGQGTLVTVSS

SEQ ID NO: 17 - protein, 119 aa, Artificial Sequence
  Description of Artificial Sequence Synthetic Peptide
EVQLVESGGGLVQPGGSLRLSCAASGFTFSNYGMSWVRQAPGKGLEWVSTISSASTYSYYPDSVKGRFTISRDNSKNTLYLQMNSLRAEDTAVYYCGRHSDGNFAFGYWGQGTLVTVSS

SEQ ID NO: 18 - protein, 119 aa, Artificial Sequence
  Description of Artificial Sequence Synthetic Peptide
EVQLVESGGGLVQPGGSLRLSCAASGFIFSNYGMSWVRQAPGKGLEWVSTISSASTYSYYPDSVKGRFTISRDNSKNTLYLQMNSLRAEDTAVYYCGRHSDGNFAFGYWGQGTLVTVSS

SEQ ID NO: 19 - protein, 112 aa, Artificial Sequence
  Description of Artificial Sequence Synthetic Peptide
DVVMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIK

SEQ ID NO: 20 - protein, 112 aa, Artificial Sequence
  Description of Artificial Sequence Synthetic Peptide
DILMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIK

SEQ ID NO: 21 - protein, 112 aa, Artificial Sequence
  Description of Artificial Sequence Synthetic Peptide
DVLMTQSPLSLPVTPGEPASISCRSSRNIVHINGDTYLEWYLQKPGQSPQLLIYKVSNRFSGVPDRFSGSGSGTDFTLKISRVEAEDVGVYYCFQGSLLPWTFGQGTKVEIK

SEQ ID NO: 22 - protein, 28 aa, Homo sapiens
NPTDIADTTLDESIYSNYYLYESIPKPC

SEQ ID NO: 23 - protein, 18 aa, Homo sapiens
DESIYSNYYLYESIPKPC

SEQ ID NO: 24 - protein, 13 aa, Homo sapiens
ESIYSNYYLYESI

(19) United States
(12) Patent Application Publication
(10) Pub. No.: US 2007/0172476 A1
(43) Pub. Date: Jul. 26, 2007

(54) Method of treating B-cell neoplasms or Hodgkin's lymphoma

(75) Inventors: Ryuzo Ueda, Aichi (JP); Takashi Ishida, Aichi (JP); Shiro Akinaga, Tokyo (JP); Toshihiko Ishii, Shizuoka (JP); Daniel M. Jones, Houston, TX (US); Richard J. Ford JR., Houston, TX (US); Jahan Khalili, Houston, TX (US); Kaushali Patel, Houston, TX (US)

Correspondence Address: FITZPATRICK CELLA HARPER & SCINTO, 30 ROCKEFELLER PLAZA, NEW YORK, NY, 10112, US

(73) Assignees: Kyowa Hakko Kogyo Co., Ltd., Chiyoda-ku (JP); Board of Regents, The University of Texas System, Austin, TX (US)

(21) Appl. No.: 11/494,499

(22) Filed: Jul. 28, 2006

Related U.S. Application Data
(60) Provisional application No. 60/702,986, filed on Jul. 28, 2005.

(51) Int. Cl. A61K 39/395

(52) U.S. Cl. 424/143.1

(57) ABSTRACT

The present invention relates to a method for injuring or depleting a B-cell neoplasm cell or a Hodgkin's lymphoma cell and a method for treating B-cell neoplasm or Hodgkin's lymphoma, wherein each method comprises administering to a patient a monoclonal antibody which specifically binds to a human CC chemokine receptor 4 (CCR4) or an antibody fragment thereof.

### FIELD OF THE INVENTION

The present invention relates to a method for injuring or depleting a B-cell neoplasm cell or a Hodgkin's lymphoma cell, which comprises administering to a patient a monoclonal antibody which specifically binds to a human CC chemokine receptor 4 (CCR4) or an antibody fragment thereof. Also, the present invention relates to a method for treating B-cell neoplasm or Hodgkin's lymphoma, which comprises administering to a patient a monoclonal antibody which specifically binds to a human CC chemokine receptor 4 (CCR4) or an antibody fragment thereof

### BACKGROUND OF THE INVENTION

When a ligand binds to a chemokine receptor, leukocyte migration is induced. A human CC chemokine receptor 4 (hereinafter referred to as “CCR4”) which is expressed mainly on Th2 type CD4-positive helper T cell (hereinafter sometimes referred to as “Th2 cell”) in normal tissues is one of the chemokine receptor family (Journal of Experimental Medicine, 187, 129-34 (1998)). CCR4 specifically binds to TARC (thymus and activation-regulated chemokine) or MDC (macrophage-derived chemokine) which are ligands for CCR4. Th2 cell is a regulatory cell in humoral immunity. While the Th2 cell promotes the production of antibodies for extraneous antigens by B cell, the Th2 cell also suppresses Th1 cell which is another subset in the helper T cell through the production of immunosuppressive cytokines such as interleukin (IL)-10, to thereby suppress cellular immunity (induction of antigen-specific cytotoxic T cell).

Since an antibody has high binding activity and binding specificity and high stability in blood, it has been attempted to apply to diagnosis, prevention and treatment of various human diseases [Monoclonal Antibodies: Principles and Applications, Wiley-Liss, Inc., Chapter 2.1 (1995)]. It has been also attempted to prepare a humanized antibody such as a human chimeric antibody or a human complementarity-determining region (hereinafter referred to as “CDR”)-grafted antibody from an antibody derived from non-human animals utilizing the genetic recombination techniques. The human chimeric antibody is an antibody in which its antibody variable region (hereinafter referred to as “V region”) is an antibody of non-human animal and its constant region (hereinafter referred to as “C region”) is a human antibody. The human CDR-grafted antibody is an antibody in which CDR of human antibody is replaced by CDR of antibody of non-human animals.

It has been known that, there are five classes in antibodies of mammals, namely IgM, IgD, IgG, IgA and IgE and, with regard to diagnosis, prevention and treatment of various diseases of human being, antibody of human IgG class has been mainly utilized because of its functional characteristics such as half-life in blood is long and various effector functions are available [Monoclonal Antibodies: Principles and Applications, Wiley-Liss, Inc., Chapter 1 (1995)]. Antibodies of human IgG class are further classified into four subclasses of IgG1, IgG2, IgG3 and IgG4. Studies for effector functions of IgG class antibodies such as antibody-dependent cell-mediated cytotoxic activity (hereinafter referred to as “ADCC activity”) and complement-dependent cytotoxic activity (hereinafter referred to as “CDC activity”) have been carried out so far and it has been reported that, in the human IgG class, antibody of the IgG1 subclass has the highest ADCC activity and CDC activity [Chemical Immunology, 65, 88 (1997)]. From the above facts, most of anti-tumor humanized antibodies such as an anti-CD20 chimeric antibody, rituximab (manufactured by IDEC/Genentech) which is an indication for non-Hodgkin's lymphoma and an anti-HER2 antibody, trastuzumab (manufactured by Roche/Genentech) which is an indication for recurrent breast cancer are antibodies of human IgGI subclass achieving a high effector function.

With regard to an example of antibodies to CCR4, a humanized antibody KM8761 of a human IgG1 type has been known (EP 1449850). It has been known that KM8761 injures normal Th2 cells or T cell leukemia/lymphoma cells via an ADCC activity (EP 1449850; Cancer Research, 64: 2127-33, 2004) and a possibility of becoming a therapeutic agent for allergy or T cell leukemia/lymphoma has been shown. However, with regard to expression of CCR4 on cell surfaces of hematopoietic tumor, only the above-mentioned T cell leukemia/lymphoma has been known and neither expression of CCR4 on cell surfaces of other cancer species nor anti-tumor effect of an anti-CCR4 antibody has been known.

### SUMMARY OF THE INVENTION

An object of the present invention is to provide an agent for injuring or depleting an in vivo B-cell neoplasm cell or an in vivo Hodgkin's lymphoma cell. Also, an object of the present invention is to provide an agent for treating B-cell neoplasm or Hodgkin's lymphoma.

The present invention relates to a method for injuring or depleting a B-cell neoplasm cell or a Hodgkin's lymphoma cell, which comprises administering to a patient a monoclonal antibody which specifically binds to a human CC chemokine receptor 4 (CCR4) or an antibody fragment thereof. Also, the present invention relates to a method for treating B-cell neoplasm or Hodgkin's lymphoma, which comprises administering to a patient a monoclonal antibody which specifically binds to a human CC chemokine receptor 4 (CCR4) or an antibody fragment thereof

### BRIEF DESCRIPTION OF THE DRAWINGS

FIG. 1 shows expression of CCR4 on the cell surface of the Hodgkin's lymphoma cell line. Left and right graphs show the results in L428 cells and in HDLM2 cells, respectively. The ordinate shows cell numbers while the abscissa shows fluorescence intensity. The histogram in gray color shows the stained result in negative control antibody mouse IgG1/κ while that in solid white shows the result in anti-CCR4 monoclonal antibody MK 2160.

FIG. 2 shows ADCC activity of anti-CCR4 human CDR-grafted antibody KM8761 to a Hodgkin's lymphoma cell line. Left and right graphs show the results when L428 cells and HDLM2 cells are used as target cells, respectively. The ordinate shows a cytotoxic activity (%) while the abscissa shows concentration (μg/mL) of anti-CCR4 human CDR-grafted antibody KM8761.

FIG. 3 shows expressions of CCR4 (left graph) and CD20 (right graph) on cell surfaces of diffuse large-cell lymphoma cell line KIS-1. The ordinate shows cell numbers while the abscissa shows fluorescence intensity. The histogram in gray color shows the stained result in negative control antibody IgG2b/κ while that in solid white shows the result in anti-CCR4 human CDR-grafted antibody KM8761 or an anti-CD20 antibody rituximab.

FIG. 4 shows ADCC activity of the anti-CCR4 human CDR-grafted antibody KM8761 and anti-CD20 antibody rituximab to diffuse large-cell lymphoma cell line KIS-1. From left to right, results in which PBMC of three blood donors (A, B and C) was used as effector cells are shown, respectively. The ordinate shows cytotoxic activity (%) while the abscissa shows antibody concentration (μg/mL).

FIG. 5 shows the anti-tumor activity of anti-CCR4 human chimeric antibody KM2760 in diffuse large-cell lymphoma cell line KIS-1 cell-transplanted mice. The ordinate shows tumor volume (mm³) while the abscissa shows the elapse (day(s)) from the initial day of administration of the antibody.

FIG. 6 shows an anti-tumor activity of an anti-CCR4 human CDR-grafted antibody KM8761 in Hodgkin's lymphoma cell line L 428 cell-transplanted mice. The ordinate shows tumor volume (mm³) while the abscissa shows the elapse (day(s)) from the initial day of administration of the antibody.

### DETAILED DESCRIPTION OF THE INVENTION

The present invention relates to the following (1) to (16).
(1) A method for injuring or depleting a B-cell neoplasm cell, which comprises administering to a patient a monoclonal antibody which specifically binds to a human CC chemokine receptor 4 (CCR4) or an antibody fragment thereof.
(2) A method for injuring or depleting a Hodgkin's lymphoma cell, which comprises administering to a patient a monoclonal antibody which specifically binds to a human CC chemokine receptor 4 (CCR4) or an antibody fragment thereof.
(3) A method for treating B-cell neoplasm, which comprises administering to a patient a monoclonal antibody which specifically binds to a human CC chemokine receptor 4 (CCR4) or an antibody fragment thereof.
(4) A method for treating Hodgkin's lymphoma, which comprises administering to a patient a monoclonal antibody which specifically binds to a human CC chemokine receptor 4 (CCR4) or an antibody fragment thereof.
(5) The method according to any one of the above (1) to (4), wherein the monoclonal antibody which specifically binds to CCR4 is an antibody which specifically binds to an extracellular region of CCR4.
(6) The method according to the above (5), wherein the extracellular region is an extracellular region selected from the group consisting of positions 1 to 39, 98 to 112, 176 to 206 and 271 to 284 in the amino acid sequence represented by SEQ ID NO:1.
(7) The method according to (5) or (6), wherein the extracellular region comprises an amino acid sequence represented by positions 2 to 29 in the amino acid sequence represented by SEQ ID NO:1.
(8) The method according to any one of the above (5) to (7), wherein the extracellular region comprises an amino acid sequence represented by positions 13 to 25 in the amino acid sequence represented by SEQ ID NO:1.
(9) The method according to any one of the above (1) to (8), wherein the monoclonal antibody which specifically binds to CCR4 or an antibody fragment thereof has lower affinity to a peptide in which at least one tyrosine residue at positions 16, 19, 20 and 22 in a peptide comprising positions 13 to 25 in the amino acid sequence represented by SEQ ID NO:1 is sulfated, than affinity to a peptide comprising positions 13 to 25 in the amino acid sequence represented by SEQ ID NO:1.
(10) The method according to any one of the above (1) to (9), wherein the monoclonal antibody is a human chimeric antibody or a human CDR-grafted antibody.
(11) The method according to the above (9), wherein the human chimeric antibody comprises complementarity determining regions (CDRs) of a heavy chain (H chain) variable region (V region) and a light chain (L chain) V region of the monoclonal antibody which specifically binds to CCR4.
(12) The method according to the above (10) or (11), wherein the human chimeric antibody comprises CDR1, CDR2 and CDR3 in the antibody heavy chain (H chain) variable region (V region) having the amino acid sequences represented by SEQ ID NOs:2, 3 and 4, respectively, and/or CDR1, CDR2 and CDR3 in the antibody light chain (L chain) V region having the amino acid sequences represented by SEQ ID NOs:5, 6 and 7, respectively.
(13) The method according to any one of the above (10) to (12), wherein the human chimeric antibody comprises an a heavy chain (H chain) variable region (V region) of an antibody molecule consisting of the amino acid sequence represented by SEQ ID NO:8 and/or a light chain (L chain) variable region (V region) of an antibody molecule represented by SEQ ID NO:9.
(14) The method according to the above (13), wherein the human CDR-grafted antibody comprises complementarily determining regions (CDRs) of a heavy chain (H chain) variable region (V region) and a light chain (L chain) V region in the monoclonal antibody which specifically binds to CCR4.
(15) The method according to the above (10) or (14), wherein the human CDR-grafted antibody comprises CDR1, CDR2 and CDR3 in an antibody heavy chain (H chain) variable region (V region) having the amino acid sequences represented by SEQ ID NOs:2, 3 and 4, respectively, and/or CDR1, CDR2 and CDR3 in an antibody light chain (L chain) V region having the amino acid sequences represented by SEQ ID NOs:5, 6 and 7, respectively.
(16) The method according to any one of the above (10), (14) and (15), wherein the human CDR-grafted antibody comprises a heavy chain (H chain) variable region (V region) of an antibody molecule consisting of the amino acid sequence represented by SEQ ID NO:10 or 11 and/or a light chain (L chain) V region of an antibody molecule consisting of the amino acid sequence represented by SEQ ID NO:12.

The present invention will now be illustrated in detail below.

With regard to a method for injuring or depleting the B-cell neoplasm cell or the Hodgkin's lymphoma cell in the present invention, any method may be used, so long as it is a method in which the B-cell neoplasm cell or the Hodgkin's lymphoma cell existing in living body are injured or depleted by administering a monoclonal antibody which specifically binds to CCR4 or a fragment of the antibody (hereinafter referred to as an “anti-CCR4 antibody”) to a patient.

The B-cell neoplasm in the present invention includes any tumor derived from B cells. Specifically, it includes B-cell neoplasms classified under WHO Classification (Tumors of Haematopoietic and Lymphoid Tissues, IARC-Press, Lyon, 2001) and specific examples include precursor B-cell lymphoblastic leukemia/lymphoma, B-cell chronic lymphocytic leukemia, B-cell prolymphocytic leukemia, lymphoplasmacytic lymphoma, mantle cell lymphoma, follicular lymphoma, cutaneous follicle center lymphoma, marginal zone B-cell lymphoma, nodal marginal zone lymphoma, splenic marginal zone B-cell lymphoma, hairy cell leukemia, diffuse large B-cell lymphoma, Burkitt's lymphoma, plasmacytoma and plasma cell myeloma.

Hodgkin's lymphoma of the present invention includes Hodgkin's lymphoma classified by the WHO classification.

An example of a method for treating B-cell neoplasm or Hodgkin's lymphoma according to the present invention is a method for treating a patient suffering from B-cell neoplasm or Hodgkin's lymphoma in which a B-cell neoplasm cell or a Hodgkin's lymphoma cell existing in the living body is injured or depleted by using an anti-CCR4 antibody or a fragment of the antibody by administering it to a patient.

The anti-CCR4 antibody used in the present invention may be any antibody, so long as it specifically binds to CCR4 and the antibody fragment thereof. As the anti-CCR4 antibody used in the present invention, it is preferred that the monoclonal antibody can regulate function of CCR4-positive cells or can exclude CCR4-positive cells from the living body. Examples include a monoclonal antibody and the like which binds to the extracellular region of CCR4 and can injure CCR4-expressing cells by the cytotoxic activity through their binding to the extracellular region of CCR4. The cytotoxic activity includes the complement-dependent cytotoxic activity (hereinafter referred to as “CDC activity”) and the antibody-dependent cell-mediated cytotoxic activity (hereinafter referred to as “ADCC activity”). Preferably, ADCC activity is exemplified.

The monoclonal antibody used in the present invention includes any of a monoclonal antibody produced by a hybridoma, a humanized antibody such as a human chimeric antibody, human CDR-grafted antibody and the like.

It is preferred that the anti-CCR4 antibody specifically binds to the extracellular region of human CCR4. The anti-CCR4 antibody include an antibody which specifically binds to the region comprising positions 1 to 39, 98 to 112, 176 to 206 or 271 to 284 in the amino acid sequence represented by SEQ ID NO:1, and preferably an antibody which binds to positions 2 to 29 (SEQ ID NO:22) in the amino acid sequence represented by SEQ ID NO:1, more preferably an antibody which binds to positions 12 to 29 (SEQ ID NO:23) in the amino acid sequence represented by SEQ ID NO:1, and most preferably an antibody which binds to positions 13 to 25 (SEQ ID NO:24) in the amino acid sequence represented by SEQ ID NO:1. Moreover, it is preferably an antibody which does not react with a human platelet.

The anti-CCR4 antibody can be prepared by a known method (Antibodies: A Laboratory Manual).

The anti-CCR4 monoclonal antibody produced by a hybridoma can be specifically prepared by the following method.

That is, a cell which expresses the CCR4 protein or a synthetic peptide based on a partial sequence of CCR4 is prepared as an antigen, and a plasma cell having an antigen specificity is induced from an animal immunized by the antigen. Then, the plasma cell is fused with a myeloma cell to prepare a hybridoma, the hybridoma is cultured or the hybridoma cells are administered into the animal to cause ascites tumor in the animal, and an antibody which specifically binds to CCR4 is separated and purified from the culture or ascites. The thus obtained anti-CCR4 monoclonal antibody includes a monoclonal antibody KM2160 produced by a hybridoma KM2160 belonging to the mouse IgG1 subclass (EP 1270595; Int. Immunol., 11, 81 (1999)).

The humanized antibody used in the present invention can be produced by using genetic recombinant techniques.

A human chimeric antibody is an antibody comprising a heavy chain variable region (hereinafter the heavy chain, the variable region and the H chain V region are also referred to as “H chain”, “V region”, and “VH”, respectively) and a light chain V region (hereinafter the light chain and the L chain V region are also referred to as “L chain” and “VL”, respectively) of an antibody derived from a non-human animal, an H chain constant region (hereinafter the constant region and the H chain C region are also referred to as “C region” and “CH”, respectively) of a human antibody, and an L chain C region (hereinafter also referred to as “CL”) of a human antibody. The non-human animal may be any of mouse, rat, hamster, rabbit and the like, so long as a hybridoma can be prepared therefrom.

The human chimeric antibody used in the present invention can be produced by obtaining cDNAs encoding VH and VL from a hybridoma capable of producing an anti-CCR4 antibody, inserting each of the DNAs into an expression vector for animal cell having DNAs encoding CH and CL of a human antibody to construct a human chimeric antibody expression vector, and introducing the vector into an animal cell to express the antibody.

Any CH of a human chimeric antibody may be used, so long as it belongs to human immunoglobulin (hIg), but those of hIgG class are preferred, and any one of subclasses further belonging to hIgG such as γ1, γ2, γ3 and γ4 can be used. Also, any CL of a human chimeric antibody may be used, so long as it belongs to hIg, and those of κ class or λ class can be used.

The human chimeric antibody which specifically binds to CCR4 (hereinafter, also referred to as “anti-CCR4 chimeric antibody”) is preferably a human chimeric antibody which comprises:

CDR1, CDR2 and CDR3 of VH having the amino acid sequences represented by SEQ ID NOs:2, 3 and 4, respectively, and/or

CDR1, CDR2 and CDR3 of VL having the amino acid sequences represented by SEQ ID NOs:5, 6 and 7, respectively, and

is more preferably a human chimeric antibody or the antibody fragment thereof which comprises:

VH comprising the amino acid sequence represented by SEQ ID NO:8, and/or

VL comprising the amino acid sequence represented by SEQ ID NO:9.

Specifically, it includes a human chimeric antibody KM2760 or the antibody fragment thereof wherein VH and CH of the antibody comprise the amino acid sequence of SEQ ID NO:8 and the amino acid sequence of human γ1 subclass, respectively, and VL and CL of the antibody comprise the amino acid sequence of SEQ ID NO:9 and the amino acid sequence of human κ class, respectively.

The above human chimeric antibody can be produced by the conventional method such as the method disclosed in EP 127095.

Transformant KM2760 which is capable of producing a human chimeric antibody KM2760 has been internationally deposited as FERM BP-7054 on Feb. 24, 2000, in National Institute of Bioscience and Human Technology, Agency of Industrial Science and Technology, the Ministry of International Trade and Industry (present name: International Patent Organism Depositary, National Institute of Advanced Industrial Science and Technology) (Higashi 1-1-3, Tsukuba-shi, Ibaraki-ken, Japan (present address: Tsukuba Central 6, 1-1, Higashi 1-Chome, Tsukuba-shi, Ibaraki-ken, Japan)).

The human CDR-grafted antibody is an antibody in which CDRs of VH and VL of an antibody from a human are respectively substituted by CDR sequences of an antibody from a non-human animal.

The human CDR-grafted antibody used in the present invention can be produced by constructing cDNAs encoding V regions in which CDR sequences of VH and VL in any human antibody are replaced by CDR sequences of VH and VL in an anti-CCR4 antibody derived from a non-human animal, inserting them respectively into an expression vector for animal cell having gene encoding CH of a human antibody and CL of a human antibody to construct a human CDR-grafted antibody expression vector, and then introducing it into an animal cell to express the human CDR-grafted antibody.

Any CH in the human CDR-grafted antibody can be used, so long as it belongs to hIg. Preferably, an hIgG class, and any one of γ1, γ2, γ3 and γ4 subclasses belonging to the hIgG class can be used. Also, any CL in the human CDR-grafted antibody can be used, so long as it belongs to the hIg, and those of κ class or λ class can be used.

The human CDR-grafted antibody which specifically binds to CCR4 (hereinafter also referred to as “anti-CCR4 CDR-grafted antibody”) is preferably a human CDR-grafted antibody or the antibody fragment thereof which comprises:

CDR1, CDR2 and CDR3 of VH having the amino acid sequences represented by SEQ ID NOs:2, 3 and 4, respectively, and/or

CDR1, CDR2 and CDR3 of VL having the amino acid sequences represented by SEQ ID NOs:5, 6 and 7, respectively,

more preferably a human CDR-grafted antibody or the antibody fragment thereof which comprises:

VH comprising the amino acid sequence represented by SEQ ID NO:10 or 11, and/or

VL comprising the amino acid sequence represented by SEQ ID NO:12, and

still more preferably a human CDR-grafted antibody or the antibody fragment thereof which comprises:

VH comprising an amino acid sequence in which at least one amino acid residue selected from Ala at position 40, Gly at position 42, Lys at position 43, Gly at position 44, Lys at position 76 and Ala at position 97 in the amino acid sequence represented by SEQ ID NO:10 is substituted with an other amino acid, and/or

VL comprising an amino acid sequence in which at least one amino acid residue selected from Ile at position 2, Val at position 3, Gln at position 50 and Val at position 88 in the amino acid sequence represented by SEQ ID NO:12 is substituted with an other amino acid, or

a human CDR-grafted antibody or the antibody fragment thereof which comprises:

VH comprising an amino acid sequence in which at least one amino acid residue selected from Thr at position 28 and Ala at position 97 in the amino acid sequence represented by SEQ ID NO:11 is substituted with an other amino acid, and/or

VL comprising an amino acid sequence in which at least one amino acid residue selected from Ile at position 2, Val at position 3, Gln at position 50 and Val at position 88 in the amino acid sequence represented by SEQ ID NO:12 is substituted with an other amino acid.

Specifically, it includes a human CDR-grafted antibody or the antibody fragment thereof which comprises:

VH comprising the amino acid sequence selected from SEQ ID NOs:13 to 18, and/or

VL comprising the amino acid sequence selected from SEQ ID NOs:19 to 21.

More specifically, it includes a human CDR-grafted antibody or the antibody fragment thereof which comprises:

VH comprising the amino acid sequence represented by SEQ ID NO:13, and

VL comprising the amino acid sequence represented by SEQ ID NO:21; and

a human CDR-grafted antibody or the antibody fragment thereof comprises:

VH comprising the amino acid sequence represented by SEQ ID NO:14, and

VL comprising the amino acid sequence represented by SEQ ID NO:21.

A human CDR-grafted antibody comprising VH comprising the amino acid sequence represented by SEQ ID NO:13 and VL comprising the amino acid sequence represented by SEQ ID NO:21 includes a human CDR-grafted antibody KM8759 has been internationally deposited as FERM BP-8129 on Jul. 30, 2002, in International Patent Organism Depositary, National Institute of Advanced Industrial Science and Technology (Tsukuba Central 6, 1-1, Higashi 1-Chome, Tsukuba-shi, Ibaraki-ken, Japan).

A human CDR-grafted antibody comprising VH comprising the amino acid sequence represented by SEQ ID NO:14 and VL comprising the amino acid sequence represented by SEQ ID NO:21 includes a human CDR-grafted antibody KM8760 has been internationally deposited as FERM BP-8130 on Jul. 30, 2002, in International Patent Organism Depositary, National Institute of Advanced Industrial Science and Technology (Tsukuba Central 6, 1-1, Higashi 1-Chome, Tsukuba-shi, Ibaraki-ken, Japan).

The antibody used in the present invention includes an antibody which comprises the amino acid sequence in which one or more amino acid residues are deleted, substituted, inserted or added in the above-mentioned amino acid sequence and which specifically reacts with CCR4, and an antibody fragments thereof.

In the present invention, one or more amino acid deletion, substitution, insertion or addition in the amino acid sequence means that one or more amino acids are deleted, substituted, inserted or added to at one or plural positions in the amino acid sequence. The deletion, substitution, insertion or addition may be carried out in the same amino acid sequence simultaneously. Also, the amino acid residue substituted, inserted or added can be natural or non-natural. The natural amino acid residue includes L-alanine, L-asparagine, L-aspartic acid, L-glutamine, L-glutamic acid, glycine, L-histidine, L-isoleucine, L-leucine, L-lysine, L-methionine, L-phenylalanine, L-proline, L-serine, L-threonine, L-tryptophan, L-tyrosine, L-valine, L-cysteine and the like.

Thereinafter, preferred examples of amino acid residues which are capable of substituting one another are shown. The amino acid residues in the same group can be substituted with each other.
Group A: leucine, isoleucine, norleucine, valine, norvaline, alanine, 2-aminobutanoic acid, methionine, O-methylserine, t-butylglycine, t-butylalanine, cyclohexylalanine;
Group B: aspartic acid, glutamic acid, isoaspartic acid, isoglutamic acid, 2-aminoadipic acid, 2-aminosuberic acid;
Group C: asparagine, glutamine;
Group D: lysine, arginine, omithine, 2,4-diaminobutanoic acid, 2,3-diaminopropionic acid;
Group E: proline, 3-hydroxyproline, 4-hydroxyproline;
Group F: serine, threonine, homoserine;
Group G: phenylalanine, tyrosine.

The anti-CCR4 antibody used in the present invention includes an antibody fragment. The antibody fragment includes antibody fragments which specifically bind to CCR4, such as Fab (abbreviation of fragment of antigen binding), F(ab′)₂, Fab′, a single chain antibody (single chain Fv; hereinafter referred to as “scFv”), a dimerized V region fragment (Diabody), a disulfide stabilized antibody (disulfide stabilized Fv; hereinafter referred to as “dsFv”), and a peptide comprising CDR.

A Fab is an antibody fragment having a molecular weight of about 50,000 and antigen binding activity, in which about a half of the N-terminal side of H chain and the entire L chain, among fragments obtained by treating IgG with a protease, papain (cut at an amino acid residue at position 224 of the H chain), are bound together through a disulfide bond (S—S bond).

The Fab used in the present invention can be obtained by treating the anti-CCR4 antibody with a protease, papain. Also, the Fab can be produced by inserting DNA encoding Fab of the antibody into an expression vector for prokaryote or an expression vector for eukaryote, and introducing the vector into a prokaryote or eukaryote to express the Fab.

A F(ab′)₂ is an antibody fragment having a molecular weight of about 100,000 and antigen binding activity, which is slightly larger than the Fab bound via a S-S bond of the hinge region, among fragments obtained by treating IgG with a protease, pepsin.

The F(ab′)₂ used in the present invention can be obtained by treating the anti-CCR4 antibody with a protease, pepsin. Also, the F(ab′)₂ can be prepared by binding Fab′ described below via a thioether bond or a S—S bond.

A Fab′ is an antibody fragment having a molecular weight of about 50,000 and antigen binding activity, which is obtained by cutting a S—S bond of the hinge region of the above F(ab′)₂.

The Fab′ used in the present invention can be obtained by treating the above F(ab′)₂ with a reducing agent, dithiothreitol. Also, the Fab′ of the present invention can be produced by inserting DNA encoding an Fab′ of the anti-CCR4 antibody into an expression vector for prokaryote or an expression vector for eukaryote, and introducing the expression vector into a prokaryote or eukaryote to express the Fab′.

A scFv is a VH-P-VL or VL-P-VH polypeptide in which one chain VH and one chain VL are linked by using an appropriate peptide linker (P) of 12 or more residues and which has an antigen-binding activity.

The scFv used in the present invention can be produced by obtaining cDNAs encoding VH and VL of the anti-CCR4 antibody, constructing DNA encoding scFv, inserting the DNA into an expression vector for prokaryote or an expression vector for eukaryote, and then introducing the expression vector into a prokaryote or eukaryote to express the scFv.

A diabody is an antibody fragment in which scFv having the same or different antigen binding specificity forms a dimer, and has an divalent antigen binding activity to the same antigen or two specific antigen binding activity to different antigens.

The diabody used in the present invention, for example, a divalent diabody which specifically reacts with CCR4, can be produced by obtaining cDNAs encoding VH and VL of the anti-CCR4 antibody, constructing DNA encoding scFv having a polypeptide linker of 3 to 10 residues, inserting the DNA into an expression vector for prokaryote or an expression vector for eukaryote, and then introducing the expression vector into a prokaryote or eukaryote to express the diabody.

A dsFV is an antibody fragment which is obtained by binding polypeptides in which one amino acid residue of each of VH and VL is substituted with a cysteine residue and those cysteine residues are bound via a S—S bond between the cysteine residues. The amino acid residue which is substituted with a cysteine residue can be selected based on a three-dimensional structure estimation of the antibody in accordance with the method shown by Reiter et al. (Protein Engineering, 7, 697 (1994)).

The dsFv used in the present invention can be produced by obtaining cDNAs encoding VH and VL of the anti-CCR4 antibody, constructing DNA encoding dsFv, inserting the DNA into an expression vector for prokaryote or an expression vector for eukaryote, and then introducing the expression vector into a prokaryote or eukaryote to express the dsFv.

A peptide comprising CDR is an antibody fragment comprising at least one region of CDRs of VH and VL. The peptide comprising plural CDRs can be produced by binding directly to or via an appropriate peptide linker.

The peptide comprising CDR used in the present invention can be produced by constructing cDNA encoding CDR of VH and VL of the anti-CCR4 antibody, inserting the cDNA into an expression vector for prokaryote or an expression vector for eukaryote, and then by introducing the expression vector into a prokaryote or eukaryote to express the peptide. Also, the peptide comprising CDR can also be produced by a chemical synthesis method such as an Fmoc method (fluorenylmethoxycarbonyl method), a tBoc method (t-butyloxycarbonyl method), or the like.

The anti-CCR4 antibody used in the present invention includes derivatives of an antibody in which a radioisotope, a protein, an agent or the like is chemically or genetically bound to the anti-CCR4 antibody of the present invention.

The derivatives of the anti CCR4-antibody used in the present invention can be produced by chemically conjugating a radioisotope, a protein, agent or the like to the N-terminal side or C-terminal side of an H chain or an L chain of the anti-CCR4 antibody or the antibody fragment thereof to an appropriate substituent group or side chain of the antibody or antibody fragment or to a sugar chain in the antibody or antibody fragment (Antibody Engineering Handbook, edited by Osamu Kanemitsu, published by Chijin Shokan (1994)).

Also, it can be genetically produced by linking a DNA encoding the anti-CCR4 antibody or the antibody fragment thereof to other DNA encoding a protein to be bound, inserting the DNA into an expression vector, and introducing the expression vector into a host cell.

The radioisotope includes ¹³¹I, ¹²⁵I and the like, and it can be bound to the antibody by, e.g., a chloramine T method.

The agent is preferably a low molecular weight compound. Examples include anticancer agents such as alkylating agents (e.g., nitrogen mustard, cyclophosphamide), metabolic antagonists (e.g., 5-fluorouracil, methotrexate), antibiotics (e.g., daunomycin, bleomycin, mitomycin C, daunorubicin, doxorubicin), plant alkaloids (e.g., vincristine, vinblastine, vindesine), hormone drugs (e.g., tamoxifen, dexamethasone), and the like (Clinical Oncology, edited by Japanese Society of Clinical Oncology, published by Cancer and Chemotherapy (1996)); anti-inflammatory agents such as steroid agents (e.g., hydrocortisone, prednisone), non-steroidal drugs (e.g., aspirin, indometacin), immunomodulators (e.g., aurothiomalate, penicillamine), immunosuppressing agents (e.g., cyclophosphamide, azathioprine) and antihistaminic agents (e.g., chlorpheniramine maleate, clemastine) (Inflammation and Anti-inflammatory Therapy, Ishiyaku Shuppan (1982)); and the like. The method for binding daunomycin to an antibody includes a method in which daunomycin and an amino group of an antibody are bound via glutaraldehyde, a method in which an amino group of daunomycin and a carboxyl group of an antibody are bound via a water-soluble carbodiimide, and the like.

The protein is preferably cytokine which activates immune cells. Examples include human interleukin 2 (hereinafter referred to as “hIL-2”), human granulocyte macrophage colony-stimulating factor (hereinafter referred to as “hGM-CSF”), human macrophage colony-stimulating factor (hereinafter referred to as “hM-CSF”), human interleukin 12 (hereinafter referred to as “hIL-12”), and the like. Also, in order to injure cancer cells directly, a toxin such as ricin, diphtheria toxin and the like, can be used. For example, a fusion antibody with a protein can be produced by linking a cDNA encoding an antibody or antibody fragment to other cDNA encoding the protein, constructing DNA encoding the fusion antibody, inserting the DNA into an expression vector for prokaryote or an expression vector for eukaryote, and then introducing it into a prokaryote or eukaryote to express the fusion antibody.

Specific preparation methods and activity evaluation methods of the anti-CCR4 antibody used in the present invention; methods for injuring or depleting a B-cell neoplasm cell or a Hodgkin's lymphoma cell and methods for treating B-cell neoplasm or Hodgkin's lymphoma, which comprise administering to a patient the monoclonal antibody or the antibody fragment thereof are explained below.

1. Preparation Method of Anti-CCR4 Antibody

(1) Preparation of Antigen

The antigen necessary for preparing an anti-CCR4 antibody includes a cell which expresses CCR4 or a cell fraction thereof, CCR4, a partial fragment of CCR4, a peptide having a partial sequence of the amino acid sequence of CCR4, and the like.

The CCR4 and the partial fragment of CCR4 can be produced intracellularly or on the surface of the cell as such or as fission proteins, by constructing a recombinant vector in which a full length cDNA encoding CCR4 or a partial fragment thereof (J. Biol. Chem., 270, 19495 (1995)) is inserted into downstream of the promoter an appropriate vector, introducing this into a host cell, and then culturing the thus obtained CCR4-expressing cell in an appropriate medium. In addition, the peptide having a partial sequence of CCR4 can be prepared by using an amino acid synthesizer.

The full length cDNA encoding CCR4 or a partial fragment thereof can be prepared by a polymerase chain reaction (hereinafter referred to as “PCR”; Sambrook J. et al., Molecular Cloning, 3rd edition, Cold Spring Harbor Laboratory (2001) (hereinafter referred to as “Molecular Cloning, 3rd edition”), Ausubel F. M. et al., Current Protocols in Molecular Biology, John Wiley & Sons (1987-2001) (hereinafter referred to as “Current Protocols in Molecular Biology”)) by using a cDNA prepared from a CCR4-expressing cell in human peripheral blood or the like as the template.

Any host can be used, so long as it can express the gene of interest, such as bacteria, yeast, animal cells and insect cells. The bacteria include bacteria belonging to the genus Escherichia, the genus Bacillus and the like such as Escherichia coli and Bacillus subtilis. The yeast includes Saccharomyces cerevisiae, Schizosaccharomyces pombe and the like. The animal cells include human cell Namalwa cell, monkey cell COS cell, Chinese hamster cell CHO cell and the like. The insect cells include Sf9, Sf21 (manufactured by Farmingen), High Five (manufactured by Invitrogen) and the like.

As the vector to be introduced with the full length cDNA encoding CCR4 or a partial fragment thereof, any vector can be used, so long as the DNA can be inserted therein and be expressed in a host cell.

When bacteria such as Escherichia coli are used as the host, the expression vector preferably comprises a promoter, a ribosome binding sequence, a full length cDNA encoding CCR4 or a partial fragment thereof, a transcription termination sequence and, as occasion demands, a promoter regulatory sequence, and examples include commercially available pGEX-2T (manufactured by Amersham Biosciences), pET17b (manufactured by Novagen) and the like.

As the method for introducing a recombinant vector into a bacterium, any method can be used, so long as it is a method in which DNA can be introduced into bacteria, and examples include a method which uses a calcium ion (Cohen S. N. et al., Proc. Natl. Acad Sci. USA, 69, 2110-2114 (1972)), a protoplast method (Japanese Published Unexamined Patent Application No. 248394/88) and the like.

When yeasts are used as a host cell, the expression vector includes YEp13 (ATCC 37115), YEp24 (ATCC 37051), YCp50 (ATCC 37419) and the like.

As the method for introducing a recombinant vector into a yeast, any method can be used, so long as it is a method in which DNA can be introduced into yeast, and examples include electroporation (Becker D. M. and Guarente L., Methods. Enzymol., 194, 182-187 (1991)), a spheroplast method (Hinnen A. et al., Proc. Natl. Acad. Sci. USA, 84, 1929-1933 (1978)), a lithium acetate method (Ito H. et al., J. Bacteriol., 153, 163-168 (1983)) and the like.

When animal cells are used as the host, the expression vector includes pAGE107 (Japanese Published Unexamined Patent Application No. 22979/91; Miyaji H. et al., Cytotechnology, 3, 133-140 (1990)), pAGE103 (Mizukami T. and Itoh S., J. Biochem., 101, 1307-1310 (1987)) and the like.

Any promoter can be used, so long as it can be expressed in animal cells, and examples include the promoter of IE (immediate early) gene of cytomegalovirus (CMV), the promoter of SV40 or metalothionein and the like. In addition, the enhancer of IE gene of human CMV can be used together with the promoter.

As the method for introducing a recombinant vector into an animal cell, any method can be used, so long as it is a method for introducing DNA into an animal cell, and examples include electroporation (Miyaji H. et al., Cytotechnology, 3, 133-140 (1990)), a calcium phosphate method (Japanese Published Unexamined Patent Application No. 227075/90), a lipofection method (Felgner P. L. et al., Proc. Natl. Acad. Sci. USA, 84, 7413-7417 (1987)) and the like.

When insect cells are used as the host, a protein can be expressed, for example, by the method described in Current Protocols in Molecular Biology, O'Reilly et al., Baculovirus Expression Vectors: A Laboratory Manual, Oxford University Press (1994) or the like. That is, the recombinant vector and baculovirus are co-transfected into insect cells to obtain a recombinant virus in the culture supernatant of the insect cells, and then insect cells are infected with the recombinant virus, whereby the protein can be expressed.

The gene transfer vector includes pVL1392, pVL1393 (both manufactured by Pharmingen), pBlueBac4.5 (manufactured by Invitrogen) and the like.

The baculovirus includes a virus which infects Barathra insects, such as Autographa californica nuclear polyhedrosis virus.

The methods for co-transfecting the above gene transfer vector and the above baculovirus into an insect cell for the preparation of a recombinant virus include the calcium phosphate method (Japanese Published Unexamined Patent Application No. 227075/90), the lipofection method (Felgner P. L. et al., Proc. Natl. Acad. Sci. USA, 84, 7413-7417 (1987)) and the like.

Also, CCR4 can be produced by preparing a recombinant baculovirus using BaculoGold Starter Kit manufactured by Pharmingen or the like, and then infecting an insect cell such as the above Sf9, Sf21 or High Five with the recombinant virus (Bio/Technology, 6, 47 (1988)).

A full length CCR4 or a partial fragment thereof can be produced as such or as a fusion protein, by culturing the transformant obtained in the above in a medium and recovering CCR4 from the culture.

The method for culturing a transformant in a medium is carried out in accordance with a conventional method used in the culturing of hosts.

The medium used in the culturing of a transformant obtained by using a microorganism such as Escherichia coli or yeast as the host may be any of a natural medium or a synthetic medium, so long as it contains a carbon source, a nitrogen source, inorganic salts and the like which can be assimilated by the microorganism and can efficiently culture the transformant (Molecular Cloning, 3rd edition). The culturing is carried out at 15 to 40° C. for 16 to 96 hours generally under aerobic conditions such as shaking culture or submerged aeration agitation culture. During the culturing, pH is maintained at 3.0 to 9.0. The pH is adjusted by using an inorganic or organic acid, an alkali solution, urea, calcium carbonate, ammonia or the like. If necessary, antibiotics such as ampicillin and tetracycline can be added to the medium during the culturing.

The medium for culturing a transformant obtained by using animal cells as the host includes generally used RPMI 1640 medium, Eagle's MEM medium, media prepared by adding fetal bovine serum to these media and the like. The culturing is carried out at 35 to 37° C. for 3 to 7 days generally in the presence of 5% CO₂, and antibiotics such as kanamycin and penicillin can be added to the medium during the culturing, if necessary.

The medium for culturing a transformant obtained by using insect cells as the host includes generally used TNM-FH medium (manufactured by Pharmingen), Sf900IISFM (manufactured by Invitrogen), Excell400, Excell405 (both manufactured by JRH Biosciences) and the like. The culturing is carried out at 25 to 30° C. for 1 to 4 days. If necessary, antibiotics such as gentamicin can be added to the medium during the culturing.

In the above culturing of an animal cell or insect cell, if possible, a serum-free medium is preferably used in order to facilitate purification of a partial fragment of CCR4 as such or as a fusion protein thereof.

When a full length CCR4 or a partial fragment thereof is accumulated in the host cell as such or as a fusion protein thereof, the cells after completion of the culturing are centrifuged and suspended in an aqueous buffer, and the resulting cells are disrupted by an ultrasonication method, a French press method or the like to recover the protein in the centrifugation supernatant.

In addition, when an insoluble body is formed inside the cell, three-dimensional structure of its protein can be formed by solubilizing the insoluble body with a protein denaturing agent and diluting or dialyzing with a solution which does not contain the protein denaturing agent or contains the protein denaturing agent but in such a thin concentration that the protein is not denatured.

When the CCR4 or a partial fragment thereof or a fusion protein thereof is secreted extracellularly, the expressed protein can be recovered in the culture supernatant.

Isolation and purification can be carried out by using isolation operations such as solvent extraction, fractional precipitation with an organic solvent, salting out, dialysis, centrifugation, ultrafiltration, ion exchange chromatography, gel filtration chromatography, hydrophobic chromatography, affinity chromatography, reverse phase chromatography, crystallization and electrophoresis, alone or in combination.

The peptide having a partial sequence of the amino acid sequence of CCR4 can be produced by a chemical synthesis method such as an Fmoc method (fluorenylmethoxycarbonyl method) or a tBoc method (t-butoxycarbonyl method). It can also be produced by a peptide synthesizer manufactured by Advanced ChemTech, Inc., Applied Biosystems, Inc., Protein Technologies, Inc., Shimadzu Corp. or the like.

(2) Immunization of Animal and Preparation of Antibody-Producing Cell

An animal is immunized with the protein obtained in the above as the antigen. As the immunization method, the antigen may be directly administered to an animal subcutaneously, intravenously or intraperitoneally, but it is preferred to administer the antigen which is bound to a carrier protein having high antigenicity, or administer the antigen together with an appropriate adjuvant.

The carrier protein includes keyhole limpet hemocyanin, bovine serum albumin, bovine thyroglobulin and the like, and the adjuvant includes complete Freund's adjuvant, aluminum hydroxide gel, pertussis vaccine and the like.

The animal to be immunized includes non-human mammals such as rabbit, goat, mouse, rat and hamster.

The antigen is administered 3 to 10 times at an interval of 1 to 2 weeks after the first administration. Dose of the antigen is preferably 50 to 100 μg per animal. After each administration, blood is taken from the immunized animal from the venous plexus of the fundus of the eye or from a caudal vein, and specific affinity of the serum to the antigen is confirmed by enzyme immunoassay shown below (ELISA; Enzyme-Linked Immunosorbent Assay, 3rd edition, published by Igaku Shoin (1987); Antibodies: A Laboratory Manual, Chapter 14, Goding J. W., Monoclonal Antibodies: Principles and Practice, Academic Press (1996)) and the like.

The enzyme immunoassay can be carried out as follows.

An antigen protein or cells expressing the antigen protein are coated on a plate and allowed to react with a serum collected from the immunized animal as a primary antibody. After the reaction with the primary antibody, the plate is washed and a secondary antibody is added thereto. After the reaction, the antibody titer is measured by carrying out a detection reaction of the secondary antibody corresponding to the labeled substance.

The secondary antibody is an antibody capable of recognizing the primary antibody which is labeled with an enzyme such as peroxidase or a substance such as biotin. Specifically, when a mouse is used as the animal to be immunized, an antibody capable of recognizing mouse immunoglobulin is used as the secondary antibody.

Thereafter, a non-human mammal of which serum shows a sufficient antibody titer is used as the supply source of antibody-producing cells.

Three to 7 days after the final administration of the antigen, lymphocytes are excised from the immunized animal in accordance with a known method (Antibodies: A Laboratory Manual), and the lymphocytes are fused with myeloma cells.

A polyclonal antibody can be prepared by separating and purifying the serum.

A monoclonal antibody can be prepared by fusing the antibody-producing cell with a non-human mammal-derived myeloma cell to prepare a hybridoma, culturing the hybridoma or administering it to an animal to cause ascites tumor of the cell, and separating and purifying the culture medium or ascitic fluid.

The antibody-producing cell is collected from the spleen, lymph node, peripheral blood or the like of the antigen-administered non-human mammal.

(3) Preparation of Myeloma Cell

As the myeloma cell, any myeloma cell can be used, so long as it can proliferate in vitro, and examples include the 8-azaguanine-resistant mouse (BALB/c-derived) myeloma cell lines P3-X63Ag8-U1 (Kohler G. and Milstein C., Eur. J. Immunol., 6, 511-519 (1976)), SP2/0-Ag14 (Shulman M, et al., Nature, 276, 269-270 (1978)), P3-X63-Ag8653 (Kearney J. F. et al., J. Immunol., 123, 1548-1550 (1979)), P3-X63-Ag8 (Kohler G. and Milstein C., Nature, 256, 495-497 (1975)) and the like. The culturing and sub-culturing of these cell lines can be carried out according to a known method (Antibodies: A Laboratory Manual) to thereby obtain a cell number of 2×10⁷ cells or more until the time of cell fusion.

(4) Cell Fusion and Selection of Monoclonal Antibody

The antibody-producing cell and myeloma cell obtained in the above are washed, fused by adding cell-aggregating medium such as polyethylene glycol-1000 (PEG-1000) and then suspended in a medium. MEM medium, PBS (disodium hydrogenphosphate 1.83 g, potassium dihydrogenphosphate 0.21 g, sodium chloride 7.65 g, distilled water 1 liter, pH 7.2) or the like is used for washing the cells. In addition, in order to selectively obtain the fused cells of interest alone, HAT medium [prepared by adding 10⁻⁴ mol/l hypoxanthine, 1.5×10⁻⁵ mol/l thymidine and 4×10⁻⁷ mol/l aminopterin to the normal medium (RPMI 1640 medium supplemented with 1.5 mmol/l glutamine, 5×10⁻⁵ mol/l 2-mercaptoethanol, 10 g/ml gentamicin and 10% fetal bovine serum)] is used as the medium for suspending the fused cells.

After the culturing, a part of the culture supernatants is subjected to the following enzyme immunoassay to select samples which react with the antigen protein but do not react with non-antigen proteins. Subsequently, cloning is carried out by limiting dilution analysis, and a sample which shows a stable and high antibody titer by enzyme immunoassay is selected as a hybridoma cell line capable of producing a monoclonal antibody which specifically binds to CCR4.

The enzyme immunoassay is carried out in the same manner as described in the item 1(2), except that a hybridoma culture supernatant or a purified antibody obtained by a method which is described later is used as the primary antibody.

Specific binding of the monoclonal antibody and CCR4 can also be evaluated by the surface plasmon resolution (Karlsson R. et al., J. Immunol. Methods, 145, 229-240 (1991)).

(5) Preparation of Monoclonal Antibody

The monoclonal antibody can be prepared by separating and purifying it from a culture medium obtained by culturing a hybridoma cell or from an ascitic fluid obtained by inducing ascites through the intraperitoneal administration of a monoclonal antibody producing hybridoma cells to 8 to 10-week-old mice or nude mice treated with pristane [intraperitoneal administration of 0.5 ml of pristane (2,6,10,14-tetramethylpentadecane), followed by feeding for 2 weeks].

The method for separating and purifying the monoclonal antibody includes centrifugation, salting out with 40 to 50% saturation ammonium sulfate, a caprylic acid precipitation method, chromatography which uses a DEAE-Sepharose column, an anion exchange column, a protein A or G-column or a gel filtration column and the like, which may be used alone or in combination. According to this method, an IgG or IgM fraction is recovered to obtain a purified monoclonal antibody.

The subclass of the purified monoclonal antibody can be determined by using a monoclonal antibody typing kit or the like. The amount of the protein can be calculated by the Lowry method or from the absorbance at 280 nm.

The subclass of antibody means an isotype in the class, and includes IgG1, IgG2a, IgG2b and IgG3 in the case of mouse, and IgG1, IgG2, IgG3 and IgG4 in the case of human. Particularly, mouse IgG1 and IgG2a and human IgG1 types are useful in applying to medical treatments since they have CDC activity and ADCC activity.

2. Preparation Method of Anti-CCR4 Humanized Antibody

(1) Construction of Vector for Expression of Humanized Antibody

A vector for expression of humanized antibody is constructed to prepare a humanized antibody from an antibody derived from a non-human animal. The vector for expression of humanized antibody is an expression vector for animal cell into which genes encoding CH and CL of C region of a human antibody have been inserted, and is constructed by inserting each of genes encoding CH and CL of a human antibody into an expression vector for animal cell.

The C region of a human antibody can be CH and CL of any human antibody. Examples include CH of γ1 subclass, CH of γ4 subclass and CL of κ class of a human antibody, and the like. As the DNA encoding CH and CL of a human antibody, a chromosome DNA which comprises exon and intron can be used. Also, cDNA can be used. As the expression vector for animal cell, any expression vector can be used, so long as a C region of a human antibody can be inserted and expressed.

Examples include pAGE107 (Japanese Published Unexamined Patent Application No. 22979/91; Miyaji H. et al., Cytotechnology, 3, 133-140 (1990)), pAGE103 (M. Mizukami T. and Itoh S., J. Biochem., 101, 1307-1310 (1987)), pHSG274 (Brady G. et al., Gene, 27, 223-232 (1984)), pKCR (O'Hare K. et al., Proc. Natl. Acad. Sci. USA, 78, 1527-1531 (1981)), pSGIβd2-4 (Miyaji H. et al., Cytotechnology, 4, 173-180 (1990)) and the like. A promoter and enhancer used for an expression vector for animal cell include an SV40 early promoter and enhancer (Mizukami T. and Itoh S., J. Biochem., 101, 1307-1310 (1987)), a Moloney mouse leukemia virus LTR promoter and enhancer (Kuwana Y. et al., Biochem. Biophys. Res. Comun., 149, 960-968 (1987)), an immunoglobulin H chain promoter (Mason J. O. et al., Cell, 41, 479-487 (1985)) and enhancer (Gillies S. D. et al., Cell, 33, 717-728 (1983)), and the like.

The vector for expression of the humanized antibody can be either of a type in which a gene encoding an antibody H chain and a gene encoding an antibody L chain exist on separate vectors or of a type in which both genes exist on the same vector (tandem type). In respect of easiness of construction of a vector for expression of humanized antibody, easiness of introduction into animal cells, and balance between the expression amounts of antibody H and L chains in animal cells, a tandem type of the vector for expression of humanized antibody is more preferred (Shitara K. et al., J. Immunol. Methods, 167, 271-278 (1994)). The tandem type of the vector for expression of humanized antibody includes pKANTEX93 (WO97/10354), pEE18 (Bentely K. J. et al., Hybridoma, 17, 559-567 (1998)) and the like.

The constructed vector for expression of humanized antibody can be used for expression of a human chimeric antibody or a human CDR-grafted antibody in animal cells.

(2) Obtaining of cDNA Encoding VH and VL of Anti-CCR4 Antibody from Non-Human Animal

The cDNA encoding VH and VL of an anti-CCR4 antibody from a non-human animal, for example, a mouse anti-CCR4 monoclonal antibody, is prepared in the following manner.

cDNAs are synthesized by extracting mRNA from a cell capable of producing a mouse anti-CCR4 monoclonal antibody, for example., a mouse anti-CCR4 antibody producing hybridoma or the like. A cDNA library is prepared by inserting the thus synthesized cDNAs into a vector such as a phage or a plasmid. Using the C region or V region of a mouse antibody as a probe, a recombinant phage or recombinant plasmid having a cDNA encoding VH and a recombinant phage or recombinant plasmid having a cDNA encoding VL are respectively isolated from the library. By determining entire nucleotide sequences of VH and VL on the recombinant phage or recombinant plasmid, entire amino acid sequences of VH and VL are deduced from the nucleotide sequences.

As the non-human animal, any one of mouse, rat, hamster, rabbit and the like can be used, so long as a hybridoma can be prepared therefrom.

The method for preparing total RNA from a hybridoma includes the guanidine thiocyanate-cesium trifluoroacetate method (Okayama H. et al., Methods Enzymol., 154, 3-28 (1987)), and the method for preparing mRNA from the total RNA includes the oligo(dT) immobilized cellulose column method (Molecular Cloning, 3rd edition), and the like. In addition, the kit for preparing mRNA from a hybridoma includes FastTrack mRNA isolation kit (manufactured by Invitrogen), QuickPrep mRNA isolation kit (manufactured by Amersham Biosciences) and the like.

The methods for synthesizing a cDNA and preparing a cDNA library includes conventional methods (Molecular Cloning, 3rd edition; Current Protocols in Molecular Biology) or a method which uses a commercially available kit such as SuperScript Choice System for cDNA Synthesis (manufactured by Invitrogen), Zap-cDNA synthesis kit (manufactured by Stratagene) or TimeSaver cDNA synthesis kit (manufactured by Amersham Biosciences).

As the vector into which a cDNA synthesized by using a mRNA extracted from a hybridoma as the template is in preparing a cDNA library, any vector can be used, so long as the cDNA can be inserted therein. Examples include phage or plasmid vectors such as ZAP Express (manufactured by Stratagene), pBluescript II SK (+) (manufactured by Stratagene), λZAPII (manufactured by Stratagene), λgt10 (manufactured by Stratagene), λgt11 (manufactured by Stratagene), Lambda BlueMid (manufactured by Clontech), λExCell (manufactured by Amersham Biosciences), pcD2 (Okayama H. and Berg P., Mol. Cell. Biol., 3, 280-289 (1983)) and pUC18 (Yanisch-Perron C. et al., Gene, 33, 103-119 (1985)).

As the Escherichia coli into which a cDNA library constructed by a phage or plasmid vector is introduced, any Escherichia coli can be used, so long as the cDNA library can be introduced, expressed and maintained. Examples include XL1-Blue MRF′ (manufactured by Stratagene), C600 (Appleyard R. K., Genetics, 39, 440-452 (1954)), Y1088 (Young R. A. and Davis R., Science, 222, 778-782 (1983)), Y1090 (Young R. A. and Davis R., Science, 222, 778-782 (1983)), NM522 (Gough J. A. and Murray N. E., J. Mol. Biol., 166, 1-19 (1983)), K802 (Wood W. B., J. Mol. Biol., 16, 118-133 (1966)), JM105 (Yanisch-Perron C. et al., Gene, 33, 103-119 (1985)) and the like.

As the method for selecting a cDNA clone encoding VH and VL of an anti-CCR4 antibody from a non-human animal from a cDNA library, it can be selected by a colony hybridization method or plaque hybridization method (Molecular Cloning, 3rd edition) which uses an isotope- or fluorescence-labeled probe. In addition, a cDNA encoding VH and VL can also be prepared by PCR by preparing primers and using a cDNA synthesized from mRNA or a cDNA library as the template.

Nucleotide sequence of the cDNA selected by the above method can be determined by carrying out a reaction based on the dideoxy method (Sanger F. et al., Proc. Natl. Acad. Sci. USA, 74, 5463-5467 (1977)) using the cDNA cloned into an appropriate vector, and analyzing the product by using a DNA sequencer such as ABI377 (manufactured by Applied Biosystems).

(3) Analysis of Amino Acid Sequences of VH and VL of Anti-CCR4 Antibody Derived from Non-Human Animal and Identification of Amino Acid Sequence of CDR

By deducing entire amino acid sequences of VH and VL encoded by the cDNA obtained in the item 2(2) from the nucleotide sequences of the cDNA determined therein, and comparing the results with entire amino acid sequences of the VH and VL of already known antibodies (Sequences of Proteins of Immunological Interest, US Dept. Health and Human Services (1991), hereinafter referred to as “Sequences of Proteins of Immunological Interest”), whether the obtained cDNA is encoding entire amino acid sequences of VH and VL of the antibody including a secretion signal sequence can be verified. Length of the secretion signal sequence and the N-terminal amino acid sequences can be deduced and the subgroup to which they belongs can also be known, by comparing the entire amino acid sequences of VH and VL of the antibody including a secretion signal sequence with entire amino acid sequences of the VH and VL of already known antibodies (Sequences of Proteins of Immunological Interest).

In addition, novelty of the sequences can be evaluated by carrying out homology retrieval of the thus obtained entire amino acid sequences of the obtained VH and VL for any database such as SWISS-PROT or PIR-Protein by using homology search program such as BLAST (Altschul S. F. et al., J. Mol. Biol., 215, 403-410 (1990)).

The VH and VL which form the antigen binding sites of antibody comprise 4 framework regions (hereinafter referred to as “FRs”) having relatively preserved sequences and 3 CDRs (CDR1, CDR2 and CDR3) having various sequences which connect them alternately (Sequences of Proteins of Immunological Interest). The amino acid sequence of each CDR of the VH and VL can be identified by comparing it with the amino acid sequences of the V regions of already known antibodies (Sequences of Proteins of Immunological Interest).

(4) Construction of Anti-CCR4 Chimeric Antibody Expression Vector

An anti-CCR4 chimeric antibody expression vector can be constructed by inserting a cDNA encoding the VH and VL of anti-CCR4 antibody derived from a non-human animal into upstream of a gene encoding human antibody CH and CL of the vector for expression of humanized antibody which is constructed in the item 2(1). For example, a plasmid having a DNA sequence encoding the amino acid sequences of VH and VL of an anti-CCR4 antibody is obtained by amplifying the VH and VL of the antibody by PCR using a plasmid having a cDNA encoding the VH and VL of anti-CCR4 antibody derived from a non-human animal as the template and using 5′-terminal side and 3′-terminal region primers comprising nucleotide sequences encoding appropriate restriction enzyme recognizing sequences and V regions, and cloning respective amplified products in a plasmid vector such as pBluescript II SK (−) (manufactured by Stratagene) and determining their nucleotide sequences by the method described in the item 2(2). An anti-CCR4 chimeric antibody expression vector can be constructed by isolating the cDNA encoding the amino acid sequences of VH and VL of anti-CCR4 antibody from the thus obtained plasmid and cloning it into upstream of a gene encoding human antibody CH and CL of the vector for expression of humanized antibody described in the item 2 (1), in such a manner that they are expressed in an appropriate form.

(5) Construction of cDNA Encoding V Region of Anti-CCR4 CDR-Grafted Antibody

cDNAs encoding VH and VL of an anti-CCR4 CDR-grafted antibody can be obtained as follows. First, amino acid sequences of FRs in VH and VL of a human antibody to which amino acid sequences of CDRs in VH and VL of an anti-CCR4 antibody from a non-human animal antibody are grafted are selected. Any amino acid sequences of FRs in VH and VL of a human antibody can be used, so long as they are from human antibody. Examples include amino acid sequences of FRs in VH and VL of human antibodies registered in database such as Protein Data Bank, and amino acid sequences common to each subgroup of FRs in VH and VL of human antibodies (Sequences of Proteins of Immunological Interest) and the like. In order to prepare a human CDR-grafted antibody having potent activity, amino acid sequences having high homology, preferably homology of 60% or more, with amino acid sequence of FRs in VH and VL of a anti-CCR4 antibody derived from a non-human animal is preferably selected.

Then, the target amino acid sequences of CDRs in VH and VL of the anti-CCR4 antibody derived from a non-human animal are grafted to the selected amino acid sequences of FRs in VH and VL of a human antibody to design amino acid sequences of VH and VL of an anti-CCR4 CDR-grafted antibody. The designed amino acid sequences are converted to nucleotide sequences by considering the frequency of codon usage found in nucleotide sequences of genes of antibodies (Sequence of Proteins of Immunological Interest), and the nucleotide sequences encoding the amino acid sequences of VH and VL of an anti-CCR4 CDR-grafted antibody are designed. Several synthetic DNAs having a length of about 100 nucleotides are synthesized based on designed nucleotide sequences, and PCR is carried out by using them. In this case, it is preferred in each of VH and VL that 6 synthetic DNAs are designed in view of the reaction efficiency of PCR and the lengths of DNAs which can be synthesized. Furthermore, they can be easily cloned into the vector for expression of humanized antibody constructed in the item 2(1) by introducing the recognition sequence of an appropriate restriction enzyme to the 5′ end of the synthetic DNAs present on the both ends. After the PCR, an amplified product is cloned into a plasmid vector such as pBluescript SK (−) (manufactured by Stratagene), and the nucleotide sequences are determined according to the method described in the item 2(2) to obtain a plasmid having nucleotide sequences encoding VH and VL of the anti-CCR4 CDR-grafted antibody of interest.

(6) Modification of Amino Acid Sequences of VH and VL of Human CDR-Grafted Antibody

It is known that when a human CDR-grafted antibody is produced by simply grafting CDRs in VH and VL of an antibody derived from a non-human animal into FRs in VH and VL of a human antibody, its antigen-binding activity is lower than that of the original antibody from a non-human animal (Tempest P. R. et al., Bio/technology, 9, 266-271 (1991)). As the reason, it is considered that several amino acid residues in not only CDRs but also FRs directly or indirectly relate to antigen-binding activity in VH and VL of the original antibody from a non-human animal, and that they are changed to different amino acid residues of FRs in VH and VL of a human antibody. In order to solve the problem, in human CDR-grafted antibody, among the amino acid sequences of FRs in VH and VL of a human antibody, an amino acid residue which directly relates to binding to an antigen, or an amino acid residue which indirectly relates to binding to an antigen by interacting with an amino acid residue in CDR or by maintaining the three-dimensional structure of an antibody is identified and modified to an amino acid residue which is found in the original non-human animal antibody to thereby increase the antigen binding activity which has been decreased (Tempest P. R., et al., Bio/technology, 9, 266-271 (1991)). In the production of a human CDR-grafted antibody, how to efficiently identify the amino acid residues relating to the antigen binding activity in FR is most important, so that the three-dimensional structure of an antibody is constructed and analyzed by X-ray crystallography (Bernstein F. C. et al., J. Mol. Biol., 112, 535-542 (1977)), computer-modeling (Tempest P. R. et al., Protein Engineering, 7, 1501-1507 (1994)) or the like. Although the information of the three-dimensional structure of antibodies has been useful in the preparation of a human CDR-grafted antibody, no method for producing a human CDR-grafted antibody which can be applied to any antibodies has been established yet. Therefore, various attempts must be currently be necessary, for example, several modified antibodies of each antibody are prepared and the relationship between each of the modified antibodies and its antigen binding activity is examined.

Modification of amino acid residues of FR of VH and VL of a human antibody can be achieved by PCR using synthetic DNA fragments for modification as primers. A vector comprising a cDNA into which a mutation of interest was introduced (hereinafter referred to as “amino acid sequence modification vector”) is obtained by determining nucleotide sequence of the amplified product after PCR based on the method described in item 2(2) to thereby confirm that the modification of interest has been carried out.

In addition, in the case of a modification of a narrow range of amino acid sequence, it can be carried out by a PCR mutation introducing method using mutation introducing primers each comprising 20 to 35 bases. Specifically, a sense mutation primer and an antisense mutation primer, each comprising 20 to 35 bases and comprising a DNA sequence encoding the amino acid residues after modification, are synthesized, and two steps of PCR are carried out by using a plasmid comprising cDNA encoding the amino acid sequences of VH and VL to be modified as the template. By subcloning the finally amplified fragment into an appropriate vector and determining its nucleotide sequence, an amino acid sequence modification vector containing a cDNA into which the mutation of interest was introduced is obtained.

(7) Construction of Anti-CCR4 CDR-Grafted Antibody Expression Vector

An anti-CCR4 CDR-grafted antibody expression vector can be constructed by cloning cDNAs encoding VH and VL of the anti-CCR4 CDR-grafted antibody constructed in the items 2(5) and 2(6) into upstream of the DNAs encoding CH and CL of the human antibody in the vector for expression of humanized antibody as described in the item 2(1). For example, when recognition sites for an appropriate restriction enzymes are introduced to the 5′-terminal of synthetic DNAs positioned at both ends among synthetic DNAs used in the construction of VH and VL of the anti-CCR4 CDR-grafted antibody in the items 2(5) and 2(6), cloning can be carried out so that they are expressed in an appropriate form in upstream of DNAs encoding CH and CL of the human antibody in the vector for expression of humanized antibody as described in the item 2(1).

(8) Transient Expression and Activity Evaluation of Humanized Antibody

In order to efficiently evaluate the antigen binding activity of various humanized antibodies produced, the humanized antibodies can be expressed transiently by using the anti-CCR4 chimeric antibody expression vector described in the item 2(4), the anti-CCR4 CDR-grafted antibody expression vector as described in the item 2(7) or the modified expression vector thereof Any cell can be used as a host cell, so long as the host cell can express a humanized antibody. Generally, COS-7 cell (ATCC CRL1651) is used in view of its high expression amount (Warr G. W. et al, Methods in Nucleic Acids Res., CRC Press, 283 (1990)). The method for introducing the expression vector into COS-7 cell includes a DEAE-dextran method (Warr G. W. et al., Methods in Nucleic Acids Res., CRC Press, 283 (1990)), a lipofection method (Felgner P. L. et al., Proc. Natl. Acad. Sci. USA, 84, 7413-7417 (1987)), and the like.

After introduction of the expression vector, the expression amount and antigen binding activity of the humanized antibody in the culture supernatant can be measured by the enzyme immunoassay described in the item 1(2) using the culture supernatant as the primary antibody and a labeled anti-human immunoglobulin antibody as the secondary antibody.

(9) Stable Expression and Activity Evaluation of Humanized Antibody

A transformant which produces a humanized antibody stably can be obtained by introducing the anti-CCR4 chimeric antibody expression vector described in the item 2(4) or the anti-CCR4 CDR-grafted antibody expression vector described in the item 2(7) into an appropriate host cell.

The method for introducing the expression vector into a host cell includes electroporation (Japanese Published Unexamined Patent Application No. 257891/90; Miyaji H. et al., Cytotechnology, 3, 133-140 (1990)) and the like.

Any cell can be used as the host cell into which the anti-CCR4 chimeric antibody expression vector or the anti-CCR4 CDR-grafted antibody expression vector is to be introduced, so long as it can express a humanized antibody. Examples include mouse SP2/0-Ag14 cell (ATCC CRL1581), mouse P3X63-Ag8.653 cell (ATCC CRL1580), CHO cell in which a dihydrofolate reductase gene (hereinafter referred to as “DHFR gene”) is deleted (Urlaub G. and Chasin L. A., Proc. Natl. Acad. Sci. USA., 77 4216-4220 (1980)), rat YB2/3HL.P2.G11.16Ag.20 cell (ATCC No: CRL1662, hereinafter referred to as “YB2/0 cell”) and the like. In order to express a humanized antibody with high ADCC activity, a cell resistant to a lectin which recognizes a sugar chain structure in which 1-position of fucose is bound to 6-position of N-acetylglucosamine in the reducing end through α-bond in a complex type N-glycoside-linked sugar chain, for example, a host cell in which a genome is modified so as to have deleted activity of an enzyme relating to the synthesis of an intracellular sugar nucleotide, GDP-fucose, and a cell in which a genome is modified so as to have deleted activity of an enzyme relating to the modification of a sugar chain in which 1-position of fucose is bound to 6-position of N-acetylglucosamine in the reducing end through α-bond in a complex type N-glycoside-linked sugar chain are preferred. Specifically, a host cell prepared by knocking out the gene encoding α-1,6-fucosyltransferase in the host cell (WO 02/31140, WO 03/85107) is more preferable.

After introduction of the expression vector, transformants which express a humanized antibody stably are selected by culturing in a medium for animal cell culture containing an agent such as G418 (G418 sulfate; manufactured by Sigma Aldrich) (Shitara K. et al., J. Immuol. Methods, 167, 271-278 (1994)). The medium for animal cell culture includes RPMI1640 medium (manufactured by Nissui Pharmaceutical), GIT medium (manufactured by Nihon Pharmaceutical), EX-CELL302 medium (manufactured by JRH Biosciences), IMDM medium (manufactured by Invitrogen), Hybridoma-SFM medium (manufactured by Invitrogen), media obtained by adding various additives such as fetal bovine serum (FBS) to these media, and the like. The humanized antibody can be expressed and accumulated in a culture supernatant by culturing the obtained transformants in a medium. The amount of humanized antibody to be expressed and antigen binding activity of the humanized antibody in the culture supernatant can be measured by ELISA or the like described in the above item 1(4). Also, in the transformant, the amount of the humanized antibody to be produced can be increased by using a DHFR gene amplification system or the like (J. Immuol. Methods, 167, 271-278 (1994)).

The humanized antibody can be purified from the culture supernatant of the transformant by using a protein A column (Antibodies: A Laboratory Manual, Chapter 8, Goding J. W., Monoclonal Antibodies: Principles and Practice, Academic Press (1996)). Any other conventional methods for protein purification can be used. For example, the humanized antibody can be purified by a combination of gel filtration, ion-exchange chromatography, ultrafiltration and the like. The molecular weight of the H chain or the L chain of the purified humanized antibody or the antibody molecule as a whole can be measured by polyacrylamide gel electrophoresis (SDS-PAGE; laemmli U. K., Nature, 227, 680-685 (1970)), Western blotting (Antibodies: A Laboratory Manual, Chapter 12; Goding J. W., Monoclonal Antibodies: Principles and Practice, Academic Press Limited (1996)) and the like.

Antigen binding activity of the purified humanized antibody can be measured by the above enzyme immunoassay described in the item 1(2) which uses a purified humanized antibody as the primary antibody, and a labeled anti-human immunoglobulin antibody as the secondary antibody, and an immunofluorescent method (Cancer Immunol. Immunother., 36, 373 (1993)), a surface plasmon resonance which uses BIAcore™ or the like (Karlsson R. et al., J. Immunol. Methods, 145, 229-240 (1991)) and the like. The cytotoxic activity upon antigen-positive cultured cells can be evaluated by measuring CDC activity, ADCC activity and the like (Cancer Immunol. Immunother., 36, 373 (1993)). Change in the amount of produced cytokine can be measured by the ELISA, immunofluorescent method and the like.

3. Preparation of Antibody Fragment

The antibody fragment used in the present invention can be prepared based on the anti-CCR4 monoclonal antibody and the anti-CCR4 humanized antibody described in the items 1 and 2 by genetic engineering techniques or protein chemical techniques. The antibody fragment used in the present invention includes Fab, F(ab′)₂, Fab′, scFv, Diabody, dsFv, a peptide comprising CDR, and the like.

(1) Preparation of Fab

Fab can be prepared by treating an anti-CCR4 antibody with a protease, papain. After the papain treatment, when the original antibody is an IgG subclass having a protein A binding activity, uniform Fab can be recovered by separating it from IgG molecules and Fc fragments by passing through a protein A column (Goding J. W., Monoclonal Antibodies: Principles and Practice, Academic Press (1996)). When the original antibody is an antibody of IgG subclass having no protein A binding activity, Fab can be recovered by ion exchange chromatography in a fraction eluted at a low salt concentration (Goding J. W., Monoclonal Antibodies: Principles and Practice, Academic Press (1996)). In addition, Fab can also be prepared by genetic engineering techniques using Escherichia coli. For example, an Fab expression vector can be prepared by cloning the DNA encoding the antibody V region described in the items 2(2), 2(5) and 2(6) into a vector for Fab expression. As the vector for Fab expression, any vector can be used, so long as a DNA for Fab can be inserted and expressed. Examples include pIT106 (Betler M. et al., Science, 240, 1041-1043 (1988)) and the like. Fab can be formed and accumulated in an inclusion body or periplasmic space by introducing the Fab expression vector into an appropriate Escherichia coli. Active Fab can be obtained from the inclusion body by a refolding method generally used for protein, and when it is expressed in the periplasm, active Fab is leaked in the culture supernatant. Uniform Fab can be purified after the refolding or from the culture supernatant using an antigen-linked column (Borrebeck K., Antibody Engineering, A Practical Guide, Oxford University Press (1991)).

(2) Preparation of F(ab′)₂

F(ab′)₂ can be prepared by treating an anti-CCR4 antibody with a protease, pepsin. After the pepsin treatment, it can be recovered as uniform F(ab′)₂ by a purification procedure similar to the case of Fab (Goding J. W., Monoclonal Antibodies: Principles and Practice, Academic Press (1996)). In addition, it can also be prepared by the method described in the item 3(3) in which Fab′ is treated with maleimide such as N,N′-o-phenylenedimaleimide or bismaleimide hexane to form a thioether bond, or a method in which it is treated with 5,5′-dithiobis(2-nitrobenzoic acid) (DTNB) to form a disulfide bond (MaCafferty J. et al., Antibody Engineering, A Practical Approach, IRL PRESS (1996)).

(3) Preparation of Fab′

Fab′ can be obtained by treating the F(ab′)₂ described in the item 3(2) with a reducing agent such as dithiothreitol. Also, Fab′ can be prepared by genetic engineering techniques using Escherichia coli. For example, an Fab′ expression vector can be prepared by cloning the DNA encoding the antibody V region of the antibody described in the items 2(2), 2(5) and 2(6) into a vector for expression of Fab′. As the vector for expression of Fab′, any vector can be used, so long as a DNA encoding V region of the antibody described in the items 2(2), 2(5) and 2(6) can be inserted and expressed. Examples include pAK19 (Carter P. et al., Bio/technology, 10, 163-167 (1992)) and the like. Fab′ can be formed and accumulated in an inclusion body or periplasmic space by introducing the Fab′ expression vector into an appropriate Escherichia coli. Active Fab′ can be obtained from the inclusion body by a refolding method generally used for protein, and when it is expressed in the periplasmic space, it can be recovered into extracellular moiety by disrupting the cells with a treatment such as lysozyme partial digestion, osmotic pressure shock sonication or the like. Uniform Fab′ can be purified after the refolding or from the disrupted cell suspension using a protein G column or the like (MaCafferty J. et al., Antibody Engineering, A Practical Approach, IRL PRESS (1996)).

(4) Preparation of scFv

scFv can be prepared using a phage or Escherichia coli by genetic engineering techniques. For example, a DNA encoding scFv is produced by ligating DNAs encoding the VH and VL of antibody described in the items 2(2), 2(5) and 2(6) via a DNA encoding a polypeptide linker comprising an amino acid sequence of 12 residues or more. It is important that the polypeptide linker is optimized so that its addition does not interfere the binding of VH and VL to the antigen. For example, the polypeptide linker shown by Pantoliano et al. (Pantorliano M. W. et al, Biochemistry, 30, 10117-10125 (1991)) and the modified one can be used.

A scFv expression vector can be constructed by cloning the resulting DNA into a vector for expression of scFv. As the vector for scFv expression, any vector can be used, so long as a DNA for scFv can be inserted and expressed. Examples include pCANTAB5E (manufactured by Amasham Biosciences), Phfa (Lah M. et al., Hum. Antibodies Hybridomas, 5, 48-56 (1994)) and the like. The scFv expression vector was introduced into an appropriate Escherichia coli and infected with a helper phage to thereby obtain a phage which expresses scFv on the phage surface in a fused form with the phage surface protein. Also, scFv can be formed and accumulated in the inclusion body or periplasmic space of Escherichia coli into which scFv expression vector is introduced. Active scFv can be obtained from the inclusion body by a refolding method generally used for protein, and when it is expressed in the periplasmic space, it can be recovered extracellularly by disrupting the cells with a treatment such as lysozyme partial digestion, osmotic pressure shock, sonication or the like. Uniform scFv can be purified after the refolding or from the disrupted cell suspension by cation exchange chromatography or the like (McCafferty J. et al., Antibody Engineering, A Practical Approach, IRL PRESS (1996)).

(5) Preparation of Diabody

Diabody can be prepared by changing the size of the polypeptide linker for preparing the above-mentioned scFv to about 3 to 10 residues. A divalent diabody can be prepared when VH and VL of an antibody is used, and a diabody having two different specificity can be prepared when VH and VL of two antibodies which reacts with two different antigens are used (Le Gall F. et al., FEBS Letters, 453, 164-168 (1999), Courage C. et al., Int. J. Cancer, 77, 763-768 (1998)).

(6) Preparation of dsFv

dsFv can be prepared using Escherichia coli by genetic engineering techniques. First, DNAs in which an encoded amino acid residue is replaced with a cysteine residue are prepared by introducing mutation into appropriate positions of the DNAs encoding the VH and VL of antibody described in the items 2(2), 2(5) and 2(6). The modification of the amino acid residue to a cystein residue can be carried out by the mutation introduction method using PCR as described in the item 2(6). The expression vectors of VH and VL expression vectors can be prepared by cloning each of the resulting DNAs into a vector for expression of dsFv. As the vector for expression of dsFv, any vector can be used, so long as a DNA for dsFv can be inserted and expressed. Examples include pULI9 (Reiter Y. et al., Protein Engineering, 7, 697-704 (1994)) and the like. The expression vectors of VH and VL are introduced into an appropriate Escherichia coli to thereby form and accumulate the VH and VL in the inclusion body or periplasmic space. The VH and VL are obtained from the inclusion body or periplasmic space and mixed, and active dsFv can be obtained by forming a disulfide bond according to a refolding method generally used for protein. After the refolding, it can be further purified by ion exchange chromatography and gel filtration or the like (Reiter Y. et al., Protein Engineering, 7, 697-704 (1994)).

(7) Preparation of a CDR-Containing Peptide

A CDR-containing peptide can be prepared by a chemical synthesis method such as Fmoc, tBoc or the like. Also, a DNA encoding a CDR-containing peptide is prepared, and the resulting DNA is cloned into an appropriate vector for expression to thereby prepare the CDR-containing peptide expression vector. As the vector for expression, any vector can be used, so long as a DNA encoding a CDR-containing peptide can be inserted and expressed. Examples include pLEX (manufactured by Invitrogen), pAX4a+ (manufactured by MoBiTec) and the like. The expression vector is introduced into an appropriate Escherichia coli so that the CDR-containing peptide can be formed and accumulated in the inclusion body or periplasmic space. The CDR-containing peptide can be obtained from the inclusion body or periplasmic space, and it can be purified by ion exchange chromatography and gel filtration or the like (Reiter Y. et al., Protein Engineering, 7, 697-704 (1994)).

(8) Evaluation of Activity

The antigen binding activity of the above antibody fragments can be measured by the enzyme immunoassay described in the item 1(2) which uses an antibody fragment as the primary antibody, the surface plasmon resonance (Karlsson R. et al., J. Immunol. Methods, 145, 229-240 (1991)) and the like.

4. The Treatment Method of the Present Invention

The anti-CCR4 antibody used in the present invention binds to the CCR4 distributing on the cell membrane surface of a B-cell neoplasm cell or a Hodgkin's lymphoma cell which is a CCR4 expressing cell infiltrated in peripheral blood or an inflammatory site.

When the anti-CCR4 antibody used in the present invention binds on the cell surface, the B-cell neoplasm cell or the Hodgkin's lymphoma cell is injured or depleted due to the CDC activity or ADCC activity of the anti-CCR4 antibody.

Accordingly, since the B-cell neoplasm cell or the Hodgkin's lymphoma cell which is a CCR4 expressing cell is injured or depleted by administering the anti-CCR4 antibody used in the present invention in vivo, B-cell neoplasm or Hodgkin's lymphoma can be treated.

5. The Therapeutic Agent of the Present Invention

Since most part of amino acids consisted by a humanized antibody is derived from the amino acid sequence of a human antibody in comparison with the case of a monoclonal antibody derived from a non-human animal, it is expected to show high efficacy in the human body with low immunogenicity, and the effects are maintained over a long period of time. Thus, it is preferred as a preventive agent and a therapeutic agent.

The medicament comprising the anti-CCR4 antibody can be administered as a preventive agent or a therapeutic agent alone, but it is generally preferred to provide it in the form of a pharmaceutical formulation produced by mixing it with one or more pharmaceutically acceptable carriers in accordance with a method well known in the technical field of pharmaceutics.

It is preferred to select a route of administration which is the most effective in carrying out the intended treatment such as oral administration or parenteral administration, such as intraoral, tracheobronchial, intrarectal subcutaneous, intramuscular, intraarticular intravenous, and the like. Intraarticular administration and intravenous administration are preferred in case of an antibody formulation.

The dosage form includes sprays, capsules, tablets, granules, syrups, emulsions, suppositories, injections, ointments, tapes, and the like.

Preparations suitable for oral administration include emulsions, syrups, capsules, tablets, powders, granules, and the like.

Liquid preparations such as emulsions and syrups, can be produced using, additives, water; saccharides such as sucrose, sorbitol and fructose; glycols such as polyethylene glycol and propylene glycol; oils such as sesame oil, olive oil and soybean oil; antiseptics such as p-hydroxybenzoate; and flavors such as strawberry flavor and peppermint.

Capsules, tablets, powders, granules and the like can be produced using, as additives, excipients such as lactose, glucose, sucrose and mannitol; disintegrating agents such as starch and sodium alginate; lubricants such as magnesium stearate and talc; binders such as polyvinyl alcohol, hydroxypropylcellulose and gelatin; surfactants such as fatty acid esters; and plasticizers such as glycerine.

Preparations suitable for parenteral administration include injections, suppositories, sprays, and the like.

Injections can be prepared using a carrier such as a salt solution, glucose solution or a mixture thereof, or the like.

Suppositories can be prepared using a carrier such as cacao butter, hydrogenated fat, a carboxylic acid, or the like.

Also, sprays can be prepared by using the antibody or the antibody fragment as such, or in combination with a carrier or the like which does not stimulate oral and airway mucous membranes of patients and can facilitate absorption of the antibody or the antibody fragment by dispersing it as minute particles.

The carriers include lactose, glycerine, and the like. Depending on the properties of the antibody and the carrier to be used, preparations such as aerosols, dry powders and the like can be produced. The additives exemplified in the oral preparations can also be added to the parenteral preparations.

The dose and frequency of administration vary depending on intended therapeutic effect, administration method, treating period, age, body weight and the like, but the dose is generally from 10 μg/kg to 20 mg/kg per day per adult.

As the dosage forms and route of administration in administering the anti-CCR4 antibody to a model animal, they can be appropriately selected according to the properties and seriousness of the symptom of the model animal to be tested. For example, the antibody can be administered to the model animal orally or parenterally (intraperitoneal, intravenous, intraarticular, intramuscular, subcutaneous administration and the like), as such or together with other pharmaceutically acceptable carrier, filler, diluent and the like.

The mixing amount and dose of the anti-CCR4 antibody to be administered to a model animal are not particularly limited but are separately decided depending on administration method of the pharmaceutical preparation, administration form, purpose for use, specific symptoms of each model animal, body weight of the model animal and the like. The dose may be approximately from 1 μg/kg to 100 mg/kg per day and the administration interval may be approximately once a day, but it can also be administered by dividing the daily dose into 2 to 4 doses per day or more. In addition, it is also possible to administer continuously for example by drip infusion or the like. When administered to a topical part such as a joint, a dose of roughly 1 pg to 100 mg is administered per one region.

The present invention is described in the following Examples, but the present invention is not limited thereto.

### EXAMPLE 1

Analysis of Expression of CCR4 in Hodgkin's Lymphoma Cells:

Formalin-fixed slices of tumor tissues of 122 patients suffering from Hodgkin's lymphoma were used for examining the expression of CCR4. The slice was immersed in 10 mM citrate buffer (pH 6) and boiled for 15 minutes using a microwave oven (600 watts). It was then allowed to react at room temperature for 2 hours with an anti-CCR4 monoclonal antibody KM2160 adjusted to 3.3 μg/mL (Int. Immunol., 11, 81 (1999)) and then washed. A biotin-labeled anti-immunoglobulin antibody solution (manufactured by DAKO; attached to a DAKO LSAB+kit) was dropped and allowed to react with the above slice at room temperature for 10 minutes and a streptoavidin-labeled peroxidase solution (manufactured by DAKO; attached to a DAKO LSAB+kit) was dropped and allowed to react at room temperature for 10 minutes. After washing, a 3,3-diaminobenzidine chromogen solution (manufactured by DAKO; attached to a DAKO LSAB+kit) was dropped thereinto to develop color at room temperature for 10 minutes. Then staining with hematoxylin was further conducted for 1 minute followed by observing under a microscope whereupon stained degree of tumor cells (Reed-Sternberg cells) showing large shape was scored in four stages of 0 (negative), 1+ (10 to 25%), 2+ (50% or more) and 3+ (100%). The results are shown in the following Table 1.

TABLE 1

| Stained degree | Numbers of patients (%) |
|---|---|
| 0 | 80 (66) |
| 1+ | 19 (16) |
| 2+ | 15 (12) |
| 3+ | 8 (7) |

As shown in the above Table 1, in tumor cells of patients suffering from Hodgkin's lymphoma, there are cells in which CCR4 is expressed and, therefore, an anti-CCR4 antibody is effective as a therapeutic agent for Hodgkin's lymphoma.

### EXAMPLE 2

Analysis of Expression of CCR4 in Various Kinds of B-Cell Neoplasms:

In accordance with the method mentioned in Example 1, each of formalin-fixed tumor tissue slices of 7 patients suffering from Zap-70 positive chronic lymphocytic leukemia (bone marrow tissue, CLL (BM)-Zap70+), 8 patients suffering from Zap-70 negative chronic lymphocytic leukemia (bone marrow tissue, CLL (BM)-Zap70−), 6 patients suffering from B-cell chronic lymphocytic leukemia/small lymphocytic lymphoma (lymph node tissue, SLL/CLL (LN)), 35 patients suffering from mantle cell lymphoma (MCL), 61 patients suffering from follicular lymphoma (FL) and 43 patients suffering from B-cell large cell lymphoma (LCL-B) was stained with an anti-CCR4 monoclonal antibody KM2160. Whether the tumors were stained or not was observed under a microscope and the results as shown in the following Table 2.

TABLE 2

| | CCR4-positive case numbers/ | |
|---|---|---|
| Cancer types | Total case numbers | Positive rate (%) |
| CLL(BM)-Zap70+ | 6/7 | 86 |
| CLL(BM)-Zap70− | 1/8 | 13 |
| SLL/CLL(LN) | 3/6 | 50 |
| MCL | 4/35 | 13 |
| FL | 7/61 | 11 |
| LCL-B | 2/43 | 5 |

As shown in the above Table 2, CCR4 was expressed in tumor cells of patients suffering from various kinds of B-cell neoplasms which suggests that the anti-CCR4 antibody was effective as a therapeutic agent for B-cell neoplasms.

### EXAMPLE 3

Measurement of ADCC Activity of Anti-CCR4 Human CDR-Grafted Antibody KM8761 to Hodgkin's Lymphoma Cell Line:

(1) Analysis of Expression of CCR4 on the Surface of Hodgkin's Lymphoma Cell Line (Flow Cytometry)

Expression of CCR4 on cell surface of L428 and HDLM2 (both being purchased from Hayashibara Seibutsu Kagaku Kenkyusho) which are Hodgkin's lymphoma cell lines was measured by the following method.

On ice, 5×10⁵ cells were allowed to react for 40 minutes with 20 μg/mL FITC-labeled mouse anti-CCR4 monoclonal antibody KM2160 or 2% fetal bovine serum/1 mM EDTA/PBS containing negative control FITC-labeled mouse IgG1/κ (BD Bioscience). At that time, mouse serum (Chemicon) as a blocking agent was added so as to give its final concentration 50%. The cells were washed and suspended in 500 μL FACS Flow solution (BD Bioscience) and fluorescence intensity was measured using a flow cytometer FACS caliber (BD Bioscience).

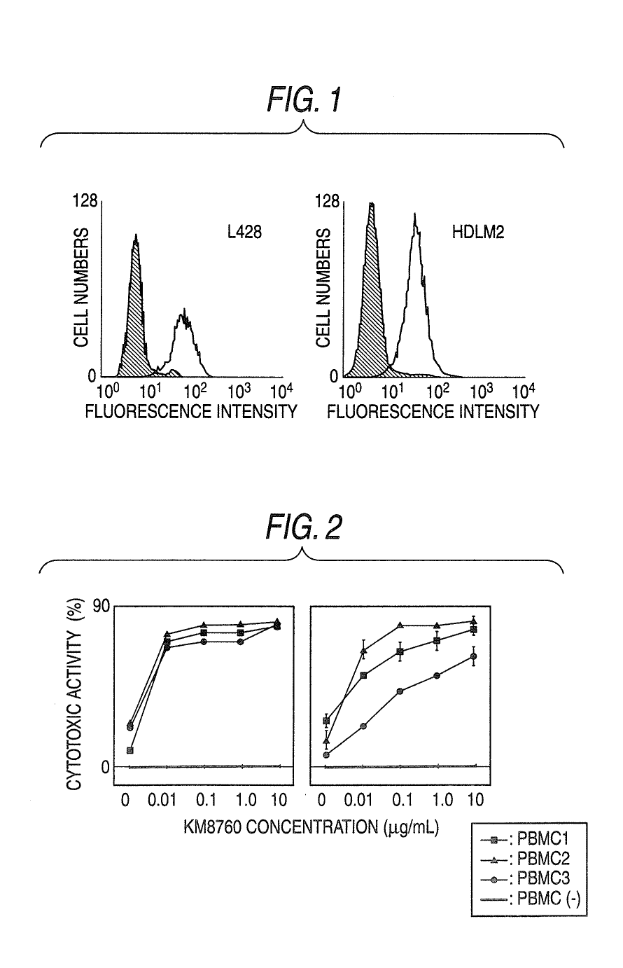

The results are shown in FIG. 1. On cell surfaces of L428 and HDLM2, clear CCR4 expression was noted.

(2) Measurement of ADCC Activity

Peripheral blood monocytes (PBMC) of three healthy volunteers were used as an effector and an ADCC activity of the anti-CCR4 human CDR-grafted antibody KM8761 to L428 and HDLM2 which are Hodgkin's lymphoma cell line in which a CCR4 expression was noted in the above item was measured by the following ⁵¹Cr liberation method.

Tumor cells (target cells) (1×10⁶ cells) were allowed to stand for 2 hours at 37° C. using 1.5 kBq of Na₂⁵¹CrO₄ to conduct incorporation of ⁵¹Cr. Natural liberation of ⁵¹Cr was promoted by being allowed to stand on ice for 15 minutes followed by washing and then cells were dispensed at 2.5×10³ cells/well/50 μL in a 96-well round-bottom plate. Further, PBMC (effector cell) separated from heparin-treated peripheral blood using Ficoll-Paque (manufactured by Pharmacia) by means of specific gravity was added thereto at 1.25×10⁵ cells/well/100 μL. Thus, the ratio of the effector cells to the target cells in terms of their numbers (E/T ratio) was 50:1. After that, 50 μL of an anti-CCR4 human CDR-grafted antibody KM8761 was added so as to give its final concentration 0, 0.01, 0.1, 1 or 10 μg/mL followed by being allowed to stand at 37° C. for 4 hours in the presence of 5% carbon dioxide. Radioactivity (cpm) of the supernatant liquid of each well was measured by a γ-counter and a cytotoxic activity was calculated by the following formula.
Cytotoxic Activity (%)=(E−S)/(M−S)×100

In the above formula 1, E is radioactivity in each experimental group, M is that in a well in which all target cells are melted by adding 1.5% of Triton X-100 and M is that by a naturally liberated ⁵¹Cr of target cells only.

The results are shown in FIG. 2. All PBMCs (PBMC-1, PBMC-2 and PBMC-3) from three donors showed an antibody concentration-dependent cytotoxic activity to two kinds of CCR4-positive Hodgkin's lymphoma cell lines. It was therefore shown that an anti-CCR4 antibody was effective as a therapeutic agent for Hodgkin's lymphoma.

### EXAMPLE 4

Measurement of ADCC Activity of Anti-CCR4 Human CDR-Grafted Antibody KM8761 to B-Cell Neoplasm Cell Line:

(1) Analysis of Expression of CCR4 and CD20 on the Surface of B-Cell Neoplasm Cell Line (Flow Cytometry)

Expression of CCR4 and CD20 on cell surface of a diffuse large B-cell lymphoma cell line, KIS-1 (Jpn. J. Cancer Res., 79, 1193-20 (1988)) was measured by the following method.

On ice, 5×10⁵ cells were allowed to react for 40 minutes with 20 μg/mL FITC-labeled mouse anti-CCR4 monoclonal antibody KM2160 or 2% fetal bovine serum/1 mM EDTA/PBS containing negative control FITC-labeled mouse IgG1/κ (manufactured by BD Bioscience). At that time, mouse serum (manufactured by Chemicon) as a blocking agent was added so as to give its final concentration 50%. The cells were washed and suspended in 500 μL FACS Flow solution (manufactured by BD Bioscience) and fluorescence intensity was measured using a flow cytometer FACS caliber (manufactured by BD Bioscience).

Similarly, KIS-1 was stained using PE-labeled anti-CD20 antibody (manufactured by BD Bioscience) and negative control PE-labeled mouse IgG2b/κ (manufactured by BD Bioscience) and intensity of fluorescence was measured using a flow cytometer.

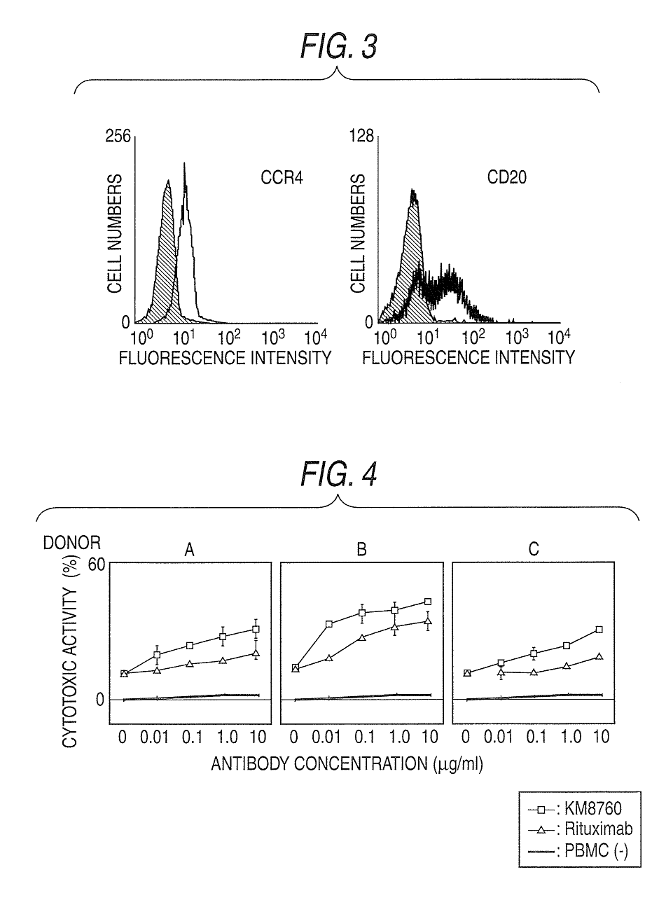

The results are shown in FIG. 3. On the cell surface of KIS-1, clear CCR4 expression and CD20 expression were noted.

(2) Measurement of ADCC Activity

Peripheral blood monocytes (PBMC) of three healthy volunteers were used as an effector and an ADCC activity of the anti-CCR4 human CDR-grafted antibody KM8761 and the anti-CD20 human IgG1/κ-type chimeric antibody rituximab (manufactured by Genentech) to a diffuse big-cell lymphoma cell line, KIS-1 in which expressions of CCR4 and CD20 were noted in the above item was measured by a method similer to that mentioned in item (2) of Example 3.

The results are shown in FIG. 4. All PBMCs derived from three donors (A, B and C) showed a cytotoxic activity to KIS-1 in a dependent manner on the concentration of anti-CCR4 human CDR-grafted antibody KM8761 which suggests that an anti-CCR4 antibody was effective as a therapeutic agent for Hodgkin's lymphoma. Further, an ADCC activity of the anti-CCR4 human CDR-grafted antibody KM8761 was higher than that of rituximab and KM8761 had a higher therapeutic effect than the conventional antibody drugs to lymphoma.

### EXAMPLE 5

Anti-Tumor Effect of an Anti-CCR4 Human Chimeric Antibody KM2760 in B-Cell Neoplasm-Transplanted Animals:

Diffuse large cell lymphoma cell line KIS-1 cells cultured in vitro were prepared into 2×10⁸ cells/mL, diluted with the same volume of Matrigel (manufactured by BD Biosciences) and 200 μL per mouse thereof was transplanted under ventral skin of a mouse (2×10⁷ cells/mouse). After 14 days from the transplantation, mice in which tumor volume was within a range of 210 to 597 mm³ (396 mm³ in average) were selected and grouped into four whereby mean value of the tumor volume became equal. Group constitution was a group to which an anti-CCR4 human chimeric antibody KM2760 was administered and a group to which no drug was administered and each group comprising five mice. From the grouped day (day 0), an anti-CCR4 human chimeric antibody KM2760 was intravenously administered once a week for four weeks. Dose per administration was made 20 mg/kg. Twice a week, long diameter (a) and short diameter (b) of the tumor were measured using a calipers and tumor volume was calculated by the following formula.
Tumor Volume (mm³)=a×b²/2

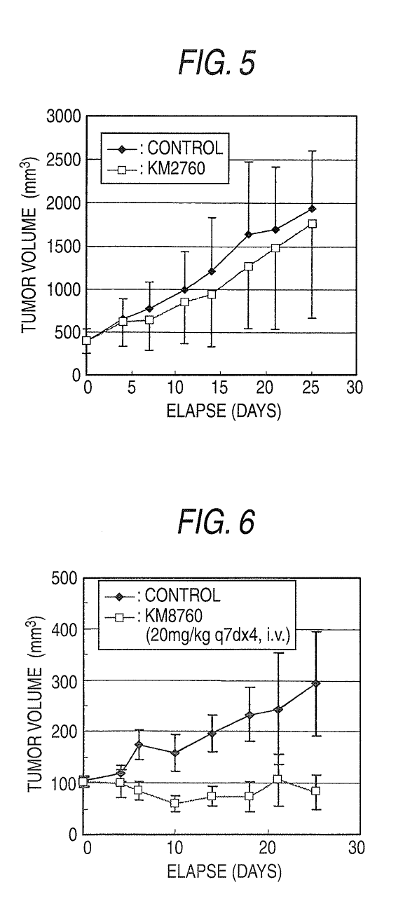

The results are shown in FIG. 5. As shown in FIG. 5, an anti-CCR4 human chimeric antibody KM2760 had an activity of suppressing the tumor.

### EXAMPLE 6

Anti-Tumor Effect of an Anti-CCR4 Human CDR-Grafted Antibody KM8761 in Hodgkin's Lymphoma Cell-Transplanted Animals:

Hodgkin's lymphoma cell line L428 cells cultured in vitro were prepared into 2×10⁷ cells/mL, diluted with the same volume of Matrigel (manufactured by BD Biosciences) and 100 μL per mouse thereof was transplanted under ventral skin of a mouse (1×10⁶ cells/mouse). After 10 days from the transplantation, mice in which tumor volume was within a range of 90 to 120 mm³ (103 mm³ in average) were selected and grouped into two whereby mean value of the tumor volume became equal. Group constitution was a group to which KM8761 was administered and a group to which no drug was administered and each group comprising five mice. From the grouped day (day 0), an anti-CCR4 human CDR-grafted antibody KM8761 was intravenously administered once a week for four weeks. Dose per administration was 20 mg/kg. Twice a week, long diameter (a) and short diameter (b) of the tumor were measured using a calipers and tumor volume was calculated by the following formula.
Tumor volume (mm³)=a×b²/2

The results are shown in FIG. 6. As shown in FIG. 6, an anti-CCR4 human CDR-grafted antibody KM8761 had an activity of suppressing the Hodgkin's lymphoma.

While the invention has been described in detail and with reference to specific embodiments thereof, it will be apparent to one skill in the art that various changes and modifications can be made therein without departing from the spirit and scope thereof.

This application is based on U.S. provisional patent application No. 60/702,986 filed Jul. 28, 2005, the entire contents of which are incorporated hereinto by reference. All references cited herein are incorporated in their entirety.

1. A method for injuring or depleting a B-cell neoplasm cell, which comprises administering to a patient a monoclonal antibody which specifically binds to a human CC chemokine receptor 4 (CCR4) or an antibody fragment thereof.

2. A method for injuring or depleting a Hodgkin's lymphoma cell, which comprises administering to a patient a monoclonal antibody which specifically binds to a human CC chemokine receptor 4 (CCR4) or an antibody fragment thereof.

3. A method for treating B-cell neoplasm, which comprises administering to a patient a monoclonal antibody which specifically binds to a human CC chemokine receptor 4 (CCR4) or an antibody fragment thereof.

4. A method for treating Hodgkin's lymphoma, which comprises administering to a patient a monoclonal antibody which specifically binds to a human CC chemokine receptor 4 (CCR4) or an antibody fragment thereof.

5. The method according to any one of claims 1 to 4, wherein the monoclonal antibody which specifically binds to CCR4 is an antibody which specifically binds to an extracellular region of CCR4.

6. The method according to claim 5, wherein the extracellular region is an extracellular region selected from the group consisting of positions 1 to 39, 98 to 112, 176 to 206 and 271 to 284 in the amino acid sequence of SEQ ID NO:1.

7. The method according to claim 5, wherein the extracellular region comprises an amino acid sequence represented by positions 2 to 29 in the amino acid sequence of SEQ ID NO:1.

8. The method according to claim 5, wherein the extracellular region comprises an amino acid sequence represented by positions 13 to 25 in the amino acid sequence of SEQ ID NO:1.

9. The method according to any one of claims 1 to 4, wherein the monoclonal antibody which specifically binds to CCR4 or an antibody fragment thereof has lower affinity to a peptide in which at least one tyrosine residue at positions 16, 19, 20 and 22 in a peptide comprising positions 13 to 25 in the amino acid sequence of SEQ ID NO:1 is sulfated, than affinity to a peptide comprising positions 13 to 25 in the amino acid sequence of SEQ ID NO:1.

10. The method according to any one of claims 1 to 4, wherein the monoclonal antibody is a human chimeric antibody or a human CDR-grafted antibody.

11. The method according to claim 10, wherein the human chimeric antibody comprises complementarity determining regions (CDRs) of a heavy chain (H chain) variable region (V region) and a light chain (L chain) V region in the monoclonal antibody which specifically binds to CCR4.

12. The method according to claim 10, wherein the human chimeric antibody comprises CDR1, CDR2 and CDR3 in the antibody heavy chain (H chain) variable region (V region) having the amino acid sequences of SEQ ID NOs:2, 3 and 4, respectively, and/or CDR1, CDR2 and CDR3 in the antibody light chain (L chain) V region having the amino acid sequences of SEQ ID NOs:5, 6 and 7, respectively.

13. The method according to claim 10, wherein the human chimeric antibody comprises an a heavy chain (H chain) variable region (V region) of an antibody molecule consisting of the amino acid sequence of SEQ ID NO:8 and/or a light chain (L chain) variable region (V region) of an antibody molecule consisting of the amino acid sequence of SEQ ID NO:9.

14. The method according to claim 10, wherein the human CDR-grafted antibody comprises complementarily determining regions (CDRs) of a heavy chain (H chain) variable region (V region) and a light chain (L chain) V region in the monoclonal antibody which specifically binds to CCR4.

15. The method according to claim 10, wherein the human CDR-grafted antibody comprises CDR1, CDR2 and CDR3 in an antibody heavy chain (H chain) variable region (V region) having the amino acid sequences of SEQ ID NOs:2, 3 and 4, respectively, and/or CDR1, CDR2 and CDR3 in an antibody light chain (L chain) V region having the amino acid sequences of SEQ ID NOs:5, 6 and 7, respectively.

16. The method according to claim 10, wherein the human CDR-grafted antibody comprises a heavy chain (H chain) variable region (V region) of an antibody molecule consisting of the amino acid sequence of SEQ ID NO:10 or 11 and/or a light chain (L chain) V region of an antibody molecule consisting of the amino acid sequence of SEQ ID NO:12.
